(19) United States
(12) Patent Application Publication
(10) Pub. No.: US 2025/0263439 A1
(43) Pub. Date: Aug. 21, 2025

(54) HETERODIMERIC PEPTIDE REAGENTS AND METHODS

(71) Applicant: REGENTS OF THE UNIVERSITY OF MICHIGAN, Ann Arbor, MI (US)

(72) Inventors: Thomas D. Wang, Ann Arbor, MI (US); Jing Chen, Ann Arbor, MI (US)

(21) Appl. No.: 19/068,888

(22) Filed: Mar. 3, 2025

Related U.S. Application Data
(63) Continuation of application No. 17/054,900, filed on Nov. 12, 2020, now abandoned.
(60) Provisional application No. 62/672,109, filed on May 16, 2018.

(51) Int. Cl. C07K 7/08; A61K 49/00

(52) U.S. Cl. CPC C07K 7/08; A61K 49/0032; A61K 49/0056

(57) ABSTRACT

The disclosure is directed to heterodimeric peptide reagents that specifically bind epidermal growth factor receptor (EGFR) and epidermal receptor growth factor 2 (ErbB2), as well as methods for detecting, targeting, diagnosing and/or treating diseases of the esophagus, including esophageal carcinoma, esophageal adenocarcinoma (EAC), high grade dysplasia (HGD) of the esophagus, and Barrett's neoplasia in a patient using the heterodimeric peptide reagents.

### STATEMENT OF GOVERNMENT INTEREST

This invention was made with government support under CA163059 and CA189291, awarded by the National Institutes of Health. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 5,923 bytes ASCII (Text) file named “52934A_SeqListing.txt,” created on May 15, 2019.

### FIELD

The disclosure is directed to heterodimeric peptide reagents as well as methods for detecting and targeting esophageal adenocarcinoma cells using the heterodimer peptide reagents.

### BACKGROUND

Esophageal adenocarcinoma (EAC) is a deadly cancer that is rising rapidly in incidence, and is associated with a poor prognosis and low five-year survival [Torre et al., Cancer Epidemiol. Biomarkers Prevent. 25: 16-27 (2016)]. Barrett's esophagus (BE) represents a metaplastic transformation of squamous into specialized columnar epithelium in response to long-standing acid and bile reflux [Spechler et al., N. Engl. J. Med. 371:836-45 (2014)]. BE is becoming more common as a result of an increasing prevalence in obesity [Whiteman et al., Gut 57(2):173-80 (2008); electronically published, Oct. 11, 2007]. High-grade dysplasia (HGD) is a pre-malignant condition that provides a window of opportunity for intervention with either curative resection or ablation therapy. [Shaheen et al., Am. J. Gastroenterol. 111: 30-50 (2016)]. Conventional endoscopic surveillance using white light illumination with random four-quadrant biopsy has been found to have limited effectiveness for localizing dysplasia that is flat in appearance, focal in extent, and patchy in distribution [Shaheen et al., supra]. Chemoprevention with nonsteroidal anti-inflammatory drugs, such as COX-2 inhibitors, has been limited by unacceptable side effects [Abrams, Therap. Adv. Gastroenterol. 1: 7-18 (2008)].

The clinical use of a peptide monomer with topical administration to detect HGD and EAC in vivo with wide-field endoscopy and confocal microendoscopy has been reported previously [Sturm et al., Sci. Transl. Med. 5: 184ra161-184ra161 (2013); Joshi et al., Endoscopy 48: A1-A13 (2016)]. Systemic delivery of the targeting ligand may improve detection of dysplastic sub-surface glands commonly seen after radio-frequency ablation (RFA) [Odze et al., Endoscopy 40: 1008-15 (2008)]. Lectins have been shown to target Barrett's neoplasia ex vivo [Bird-Lieberman et al., Nat. Med. 18: 315-21 (2012). However, these agents are low in diversity and may not have adequate binding affinity for clinical use. An Alexa Fluor 488 labeled monoclonal antibody has been administered systemically in a rat model of BE, and found heterogenous expression of ErbB2 (HER2) in EAC using confocal microendoscopy in vivo [Realdon et al., Dis. Esophagus 28: 394-403 (2015)].

Thus, new products and methods for detection and treatment of EAC, HGD, and Barrett's neoplasia are needed in the art. New products and methods would have important clinical applications for increasing the survival rate for EAC, and for reducing related healthcare costs.

### SUMMARY

The ability to identify target expression can be used to accurately diagnose, stage, and classify tumors, and to monitor their response to therapy. EAC is highly heterogeneous on gene expression profiles [Dulak et al., Cancer Res. 72: 4383-93 (2012)]. Epidermal growth factor receptor (EGFR) and epidermal growth factor receptor 2 (ErbB2) have been found to be high-frequency gene amplified and overexpressed in HGD and EAC [Dahlberg et al., Ann. Thorac. Surg. 78: 1790-1800 (2004); Cronin et al., Am. J. Gastroenterol. 106: 46-56 (2011)]. These receptor tyrosine kinases are validated cancer biomarkers and function to stimulate epithelial cell growth, proliferation, and differentiation [Citri et al., Nat. Rev. Mol. Cell Biol. 7: 505-16 (2006)]. Emerging evidence supports early expression of EGFR and ErbB2 in progression of BE to EAC when intervention can improve patient outcomes [Paterson et al., J. Pathol. 230: 118-28 (2013)]. It is contemplated herein that their location on the cell surface is well suited for development as either a diagnostic or therapeutic target. Minimal overlap in expression has been found in studies of surgically resected EAC specimens [Miller et al., Clin. Cancer Res. 9: 4819-25 (2003)]. A multiplexed approach that detects these two targets in combination is contemplated herein.

Multivalent ligands generate synergistic effects that can increase their affinity, avidity, selectivity, and potency by binding multiple targets concurrently [Rao et al., Science 280(5364): 708-11, 1998]. These properties can enhance in vivo diagnostic imaging performance by improving target-to-background (T/B) ratio and detecting targets at lower levels of expression [Luo et al., Mol. Pharm. 11: 1750-61 (2014)]. Also, this strategy can achieve additive effects for therapy by interfering with interconnected cell signaling pathways [Cochran, Sci. Transl. Med. 2: 17ps5-15, 2010]. The ability to bind multiple targets simultaneously may reduce acquired resistance that arises from prolonged binding a single target [Rosenzweig, Biochem. Pharmacol. 83: 1041-8 (2012)]. A number of bispecific antibodies have been developed for dual targeting, however effectiveness may be limited by poor tumor uptake, immunogenicity, and high manufacture costs [Wu et al., Nat. Biotechnol. 23: 1137-46 (2005)]. Seven amino acid peptide monomers specific for EGFR and ErbB2 have been validated [Zhou et al., Clin. Transl. Gastroenterol., 6(7): e101 (2015); Joshi et al., Bioconjug. Chem. 27:481-94, (2015)]. The disclosure herein combines these EGFR and ErbB2 peptide monomers in a heterodimer configuration to provide a novel and effective strategy for improved targeting performance.

The disclosure provides a reagent comprising a heterodimeric peptide comprising an epidermal growth factor receptor (EGFR)-specific peptide and an epidermal receptor growth factor 2 (ErbB2)-specific peptide, or a multimeric form of the heterodimeric peptide, wherein the heterodimeric peptide specifically binds to EGFR and ErbB2, and at least one detectable label, or at least one therapeutic moiety, or both, wherein the label, the therapeutic moiety, or both, are attached to the heterodimeric peptide or the multimeric form of the heterodimeric peptide. In some embodiments, the epidermal growth factor receptor (EGFR)-specific peptide is QRHKPRE (SEQ ID NO: 1). In some embodiments, the epidermal receptor growth factor 2 (ErbB2)-specific peptide is KSPNPRF (SEQ ID NO: 2).

In some embodiments, the EGFR-specific peptide and the ErbB2-specific peptide are joined by one or more linkers. In some aspects, the linker is attached at the C-terminus of the EGFR-specific peptide and at the C-terminus of the ErbB2-specific peptide. In some aspects, the linker has a length of about 60 Å. In some aspects, the linker is a peptide, a polyethylene glycol or an aminohexonic acid. In some aspects, the linker is triethyleneglycol (E3).

In some embodiments, at least one detectable label is attached to a peptide of the heterodimeric peptide. In some aspects, the label is attached to the peptide via a linker. In some aspects, the label is attached to the peptide by a peptide linker. In some aspects, the terminal amino acid of the peptide linker is lysine or a lysine is added at the end of the linker. In some aspects, the label is attached to the peptide by an E3 linker with a terminal lysine at the C-terminus of the E3 linker. In some aspects, the linker comprises the amino acid sequence (GGGSK) SEQ ID NO: 3, the amino acid sequence (GGGAGGG) SEQ ID NO: 28, or the amino acid sequence (GGGAGGGK) SEQ ID NO: 29.

In some aspects, the label is detectable by microscopy, photoacoustics, ultrasound, or magnetic resonance imaging. In some aspects, the label detectable by microscopy is fluorescein isothiocyanate (FITC). In some aspects, the label detectable by microscopy is Cyanine 5 (Cy5). In some aspects, the label detectable by microscopy is Cyanine 5.5 (Cy5.5). In some aspects, the label detectable by microscopy is near-infrared (NIR) fluorescent dye 800 (IRDye800).

In some embodiments, at least one therapeutic moiety is attached to the heterodimeric peptide or a peptide monomer of the heterodimeric peptide. In some aspects, the therapeutic moiety is a chemotherapeutic agent. In some aspects, the therapeutic moiety is a micelle or is provided in a micelle. In some aspects, the micelle is an octadecyl lithocholate micelle. In some aspects, the micelle is pegylated. In some aspects, the micelle comprises carboplatin and paclitaxel; cisplatin and 5-fluorouracil (f-FU); ECF: epirubicin (ELLENCE®), cisplatin, and 5-FU; DCF: docetaxel (TAXOTERE®), cisplatin, and 5-FU; cisplatin with capecitabine; oxaliplatin and either 5-FU or capecitabine; and irinotecan. In some aspects, the micelle comprises trastuzumab or ramucirumab.

In some embodiments, the disclosure provides a composition comprising a reagent comprising a heterodimeric peptide comprising an EGFR-specific peptide and an ErbB2-specific peptide, or a multimeric form of the heterodimeric peptide, wherein the heterodimeric peptide specifically binds to EGFR and ErbB2, and wherein at least one detectable label, or at least one therapeutic moiety, or both, are attached to the heterodimeric peptide or the multimeric form of the heterodimeric peptide and a pharmaceutically acceptable excipient.

In some embodiments, the disclosure provides a method for detecting esophageal adenocarcinoma (EAC), high grade dysplasia (HGD) of the esophagus, or Barrett's neoplasia in a patient comprising the steps of administering a reagent comprising a heterodimeric peptide comprising an epidermal growth factor receptor (EGFR)-specific peptide and an epidermal receptor growth factor 2 (ErbB2)-specific peptide, or a multimeric form of the heterodimeric peptide, wherein the heterodimeric peptide specifically binds to EGFR and ErbB2, and at least one detectable label, or at least one therapeutic moiety, or both, wherein the label, the therapeutic moiety, or both, are attached to the heterodimeric peptide or the multimeric form of the heterodimeric peptide to the patient and detecting binding of the reagent to esophageal cells of the patient.

In some embodiments, the disclosure provides a method of determining the effectiveness of a treatment for EAC, HGD of the esophagus, or Barrett's neoplasia in a patient comprising the step of administering a reagent comprising a heterodimeric peptide comprising an EGFR-specific peptide and an ErbB2-specific peptide, or a multimeric form of the heterodimeric peptide, wherein the heterodimeric peptide specifically binds to EGFR and ErbB2, and at least one detectable label, or at least one therapeutic moiety, or both, wherein the label, the therapeutic moiety, or both, are attached to the heterodimeric peptide or the multimeric form of the heterodimeric peptide to the patient, visualizing a first amount of cells labeled with the reagent, and comparing the first amount to a previously-visualized second amount of cells labeled with the reagent, wherein a decrease in the first amount cells labeled relative to the previously-visualized second amount of cells labeled is indicative of effective treatment. In some aspects, the method further comprises obtaining a biopsy of the cells labeled by the reagent.

In some embodiments, the disclosure provides a method for delivering a therapeutic moiety to EAC cells, HGD cells of the esophagus, or Barrett's neoplastic cells of a patient comprising the step of administering a reagent comprising a heterodimeric peptide comprising an EGFR-specific peptide and an ErbB2-specific peptide, or a multimeric form of the heterodimeric peptide, wherein the heterodimeric peptide specifically binds to EGFR and ErbB2, and at least one detectable label, or at least one therapeutic moiety, or both, wherein the label, the therapeutic moiety, or both, are attached to the heterodimeric peptide or the multimeric form of the heterodimeric peptide to the patient.

In some embodiments, the disclosure provides a kit comprising a composition comprising a reagent comprising a heterodimeric peptide comprising an EGFR-specific peptide and an ErbB2-specific peptide, or a multimeric form of the heterodimeric peptide, wherein the heterodimeric peptide specifically binds to EGFR and ErbB2, and at least one detectable label, or at least one therapeutic moiety, or both, wherein the label, the therapeutic moiety, or both, are attached to the heterodimeric peptide or the multimeric form of the heterodimeric peptide and a pharmaceutically acceptable excipient, and instructions for use of the composition in a patient or cells of a patient. In some aspects, the kit further comprises a device for administering the composition to the patient or to the cells of the patient.

The disclosure also provides uses of the reagents and uses of the heterodimeric peptides and heterodimeric peptide constructs described herein. In some aspects, these uses include, but are not limited to, diagnostics and treatment.

Other features and advantages of the disclosure will become apparent from the detailed description provided herein below. It should be understood, however, that the detailed description and the specific examples, while indicating embodiments of the disclosed subject matter, are given by way of illustration only, because various changes and modifications within the spirit and scope of the disclosure will become apparent to those skilled in the art from the description.

### BRIEF DESCRIPTION OF THE DRAWINGS

This disclosure contains at least one drawing executed in color. Copies of this patent, patent application, or patent publication with color drawing(s) will be provided by the United States Patent and Trademark Office upon request and payment of the necessary fee.

FIG. 1A-D shows the design of a peptide heterodimer of the disclosure. FIG. 1A) Chemical structures for the heptapeptide monomers QRHKPRE (SEQ ID NO: 1; also abbreviated “QRH*”), KSPNPRF (SEQ ID NO: 2; also abbreviated “KSP*”) and arranged heterodimer configuration with variable length linkers are shown. FIG. 1B) On confocal microscopy, strong binding of heterodimer was observed on the cell surface. FIG. 1C) Quantified results shows that the E3 linker gives optimal binding performance (an average of 4 images was collected independently). FIG. 1D) Western blot shows EGFR and ErbB2 overexpression on SKBr3 cells compared to QhTERT.

FIG. 2A-D shows the validation of specific binding with the heterodimer. siRNA knockdown experiments were conducted and FIG. 2A) QRH*-KSP*-E3-Cy5.5 (red) shows significantly greater binding to the surface (arrows) of siCL (control) SKBr3 cells compared with that for siEGFR (knockdown) cells. Similar results were found in siCL and siErbB2 (knockdown) cells. FIG. 2B) Quantified results are an average of five randomly chosen cells on three images collected independently. FIG. 2C) Western blot shows expression of EGFR and ErbB2 in control and knockdown cells. Apparent dissociation and association time constant were tested. FIG. 2D) The apparent dissociation constant (binding affinity) for QRH*-KSP*-E3-Cy5.5 was found to be k_{d}=23 versus 98 and 54 nM for QRH*-Cy5.5 and KSP*-Cy5.5. The apparent association time constant for QRH*-KSP*-E3-Cy5.5 was found to be Kₐ=0.22 min⁻¹ (4.5 min) versus 0.21 min⁻¹ (4.8 min) and 0.35 min⁻¹ (2.9 min) for QRH*-Cy5.5 and KSP*-Cy5.5. Results for each measurement are representative of three independent experiments.

FIG. 3A-B shows co-localization and internalization of the peptide heterodimer. FIG. 3A) Using confocal microscopy, binding was compared for QRH*-KSP*-E3-Cy5.5 (red) with that for AF568-labeled anti-EGFR antibody (yellow) and for AF488-labeled anti-ErbB2 antibody (green) to the surface (arrows) of SKBr3 cells. A Pearson's correlation coefficient of ρ=0.65 and 0.77 was measured for the heterodimer with the EGFR and ErbB2 antibodies, respectively. DAPI shows locations of cell nuclei. Results for each measurement are representative of three independent experiments. FIG. 3B) At 1-8 min after treatment, the ligand appeared to accumulate on the cell surface; at 8-20 min, the ligand began to be internalized, and at 40 min, a strong punctuate fluorescence signal appeared inside the cells (Scale bars: 25 m).

FIG. 4 shows no effect of the peptide heterodimer on cell signaling. The effect of the QRH*-KSP*-E3-Cy5.5 on downstream cell signaling was evaluated after binding to SKBr3 cells. Using Western blot, no change in phosphorylation of EGFR (p-EGFR), ErbB2 (p-ErbB2) or of downstream AKT (p-AKT) and ERK (p-ERK) was observed with incubation of the heterodimer at 1, 5, and 20 μM. By comparison, the addition of EGF, an endogenous ligand for EGFR, showed increased expression of p-AKT and p-ERK. The addition of 100 nM of lapatinib, a tyrosine kinase inhibitor known to interrupt EGFR/ErbB2 signaling in solid tumors, showed reduced expression of p-EGFR, p-ErbB2 and p-AKT. Cells treated with 1% DMSO and untreated cells showed no suppression of EGFR and ErbB2 mediated signaling. β-tubulin was used as loading control.

FIG. 5A-E shows peptide heterodimer pharmacokinetics. FIG. 5A) Representative whole-body images of NIR fluorescence show uptake of QRH*-KSP*-E3-Cy5.5 in xenograft tumor (arrows) over time. (GGGAGGG)₂KK(SEQ ID NO:7)-Cy5.5 was used as the control. FIG. 5B) Quantitative analysis shows peak mean signal from tumor at about 2 hours following intravenous injection of heterodimer in n=6 mice, and is significantly greater than that for control (n=6). Signal returns to baseline by about 24 hours. FIG. 5C) A comparison of the average tumor-fluorescence intensity at 2 h after administration of targeted (KSP*-QRH*-E3-Cy5.5, Red) and Control (Gray, n=6), P=0.0029. FIG. 5D) The addition of unlabeled QRH*, KSP*, and both QRH* and KSP* prior to heterodimer results in reduction of signal from tumor. FIG. 5E) Quantified results from n=3 mice.

FIG. 6A-D shows specific binding of the heterodimer to Barrett's neoplasia. Upon representative confocal microscopy images of human esophageal specimens ex vivo, the peptide heterodimer QRH*-KSP*-E3-Cy5.5 (red) shows minimal staining to FIG. 6A) squamous (SQ) and FIG. 6B) Barrett's esophagus (BE), and increased staining intensity with FIG. 6C) high-grade dysplasia (HGD) and FIG. 6D) esophageal adenocarcinoma (EAC). Similar results were found with AF568-labeled anti-EGFR antibody (yellow) and for AF488-labeled anti-ErbB2 antibody (green). Merged images show co-localization of peptide and antibody binding.

FIG. 7A-D shows co-localization of peptide heterodimer and antibody binding to Barrett's neoplasia. FIG. 7A) Using confocal microscopy, serial sections of HGD in human esophageal specimens are shown following staining with QRH*-KSP*-E3-Cy5.5 (red), anti-EGFR antibody labeled with AF568 (yellow) and anti-ErbB2 antibody labeled with AF488 (green). Fluorescence intensities were quantified from the mean of a set of three boxes with dimensions of 30×30 μm² placed over random crypts. Co-localization of binding is appreciated on the merged image. FIG. 7B) High-magnification images are shown from dashed boxes. On the merged image, a Pearson's correlation coefficient (ρ) was measured for the heterodimer and EGFR (ρ=0.60) and for the heterodimer and ErbB2 (ρ=0.75). FIG. 7C) From n=31, 8, 23, and 12 specimens of SQ, BE, HGD, and EAC, respectively, a significantly greater mean fluorescence intensity from HGD and EAC was found compared with that for BE and SQ with QRH*-KSP*-E3-Cy5.5. Similar results were observed for anti-EGFR-AF568 and anti-ErbB2-AF488. FIG. 7D) ROC curve shows 88% sensitivity, 87% specificity and 0.95 AUC with QRH*-KSP*-E3-Cy5.5; 74% sensitivity, 69% specificity, and 0.79 AUC with QRH*-Cy5.5; and 85% sensitivity, 79% specificity, and 0.91 AUC with KSP*-Cy5.5.

FIG. 8A-C shows mass spectrometry analysis of the monomers and heterodimers of the disclosure. The experimental mass-to-charge (m/z) ratio for Cy5.5-labeled peptide monomers and heterodimer was found to be 1794.98, 1900.04, and 2974.69 for FIG. 8A) QRH*-Cy5.5, FIG. 8B) KSP*-Cy5.5, and FIG. 8C) QRH*-KSP*-E3-Cy5.5, respectively. These results agreed with expected values. M: molecular weight. Na+: sodium ion.

FIG. 9A-B shows spectral properties of the monomers and heterodimers of the disclosure. FIG. 9A) Peak absorbance occurs at λ=680 nm and FIG. 9B) maximum fluorescence emission is seen at 708 nm for QRH*-Cy5.5, KSP*-Cy5.5, and QRH*-KSP*-E3-Cy5.5. The different peptide ligands show minimal spectral differences.

FIG. 10A-D shows the results of protein docking experiments. FIG. 10A) EGFR (1IVO) forms a homodimer prior to binding. The extracellular domain (ECD) has a head-to-head and head-to-tail dimensions of ˜60 and ˜80 Å, respectively. FIG. 10B) ErbB2 (2A91) has dimensions of ˜60 Å between domains 1 and 2. The triethyleneglycol (PEG3) linker (E3) provides the best match to the structural model, resulting in the heterodimer QRH*-KSP*-E3-Cy5.5 that binds to FIG. 10C) domain 2 of EGFR with energy Eₜ=−656.47 and FIG. 10D) domain 3 of ErbB2 with Eₜ=−632.83.

FIG. 11A-B shows heterodimer stability. At 37° C., no noticeable heterodimer degradation was observed by HPLC in either FIG. 11A) PBS containing 0.1% BSA at 0.5, 2, 6 and 12 hours or in FIG. 11B) mouse serum with 30 μM concentration at 0.5, 1.0, 1.5 and 2 hours.

FIG. 12A-C shows a comparison of binding with respect to heterodimeric peptides versus monomeric peptides to the surface of OE33 (human esophageal adenocarcinoma cells). FIG. 12A) Using confocal microscopy, a stronger binding intensity of the heterodimer was observed versus that of the monomers. QhTERT (human non-dysplastic Barrett's esophagus cells) was used as control. FIG. 12B) Quantified results represent an average of three images collected independently. FIG. 12C) Western blot for OE33 and QhTERT cells also is shown.

FIG. 13A-C shows peptide heterodimer biodistribution. FIG. 13A) Representative fluorescent images of mouse organs excised at about 2 hours following intravenous injection of QRH*-KSP*-E3-Cy5.5 in n=4 mice, and FIG. 13B) control peptide (n=4). FIG. 13C) Quantification of fluorescent signals from individual organs.

FIG. 14A-B shows immunohistochemistry (IHC) of EGFR and ErbB2 ex vivo. Sections of OE33 xenograft tumor validates expression of FIG. 14A) EGFR and FIG. 14B) ErbB2.

FIG. 15A-B shows histology (H&E) of various organs in control (FIG. 15A) or after systemic administration of the peptide heterodimer (FIG. 15B). A) PBS as control and B) QRH*-KSP*-E3-Cy5.5; the sections of mice major organs including brain, heart, kidney, liver, spleen and lung, were evaluated after day 15 and showed no sign of acute toxicity.

FIG. 16A-D displays heterodimer configuration. FIG. 16A) Chemical structure of QRH*-KSP*-E3-IRDye800 is shown. FIG. 16B) PA intensity (β-500 μg/mL) and FIG. 16C) FL emission (5-100 μg/mL) was linear over the range of heterodimer concentrations. Inset: PA and FL images of heterodimer in PBS solution at different concentrations. FIG. 16D) Negligible cytotoxicity was seen with heterodimer added to a panel of human OE19, OE21, OE33 and SKBr3 cancer cells for 48 hours at concentrations up to 500 μg/mL.

FIG. 17A-D shows cell surface binding. Strong fluorescence intensity was observed from QRH*-KSP*-E3-IRDye800 (red) binding to the surface (arrows) of FIG. 17A) SKBr3 cells. Western blot shows FIG. 17B) EGFR and FIG. 17C) HER2 expression by SKBr3, OE21, OE19, and QhTERT cells. FIG. 17D) Quantified results showed significantly greater mean (±SD) fluorescence intensity for heterodimer binding to SKBr3 (EGFR+/HER+) cells versus OE21 (EGFR+/HER−), OE19 (EGFR−/HER+), and QhTERT (EGFR−/HER−) cells by paired t-test. Results for each measurement were representative of 3 independent experiments.

FIG. 18A-D displays pharmacokinetics of the heterodimer. Representative FIG. 18A) FL and FIG. 18B) PA images are shown of tumor (arrow) at 0 (pre-injection), 1, 2, 4, and 24 hours after intravenous injection of QRH*-KSP*-E3-IRDye800. FIG. 18C) Fluorescence T/B ratio for heterodimer peaks at 2 hours post-injection. Mean (±SD) result for heterodimer (3.89±0.94) was significantly greater than that for control peptide (GGGAGGG)2-E3-IRDye800 (3.89±0.94) and PBS (1.06±0.22), P=6.4×10-4 and 3.19×10-5 by unpaired t-test with n=6 mice. FIG. 18D) Photoacoustic T/B ratio for heterodimer also peaks at 2 hours post-injection. Mean result for heterodimer (2.44±0.36) was significantly greater than that for control peptide (1.44±0.21) and PBS (1.17±0.15), P=1.6×10⁻⁴ and 1.26×10⁻⁵.

FIG. 19A-D shows biodistribution and stability. FIG. 19A) White light and FIG. 19B) FL images (λex=800 nm) are shown of heterodimer distribution in major organs at 2 hours post-injection. FIG. 19C) Mean (±SD) fluorescence intensity for heterodimer and control peptide was shown from n=6 mice each. Results from tumor were significantly greater for heterodimer versus control by an average of 2.6-fold, P=3.1×10-4 by unpaired t-test. FIG. 19D) FL intensity from serum of tumor bearing mice injected with heterodimer (300 μM, 150 μL PBS, n=6) is shown over 48 hours. Blood prior to heterodimer injection is shown as control. Quantified results showed a decrease in relative FL intensity from 90.1 to 1.3% with half-life of ˜3 hours, R2=0.95.

FIG. 20A-D shows tumor imaging depth. FIG. 20A) White light image shows location of human esophageal (OE33) xenograft tumor in a nude mouse. FIG. 20B) NIR FL image collected 2 hours after heterodimer injection shows tumor dimensions (red dash) of L×W=6.4×4.7 mm2. FIG. 20C) Sagittal view of PA image collected along (white dash) line in panel A) shows tumor (yellow dash) highlighted by heterodimer with 4.8 mm (blue) depth and 1.2 cm (red) total depth. FIG. 20D) Ultrasound image confirms tumor structure.

FIG. 21A-I shows immunofluorescence of tumor. FIG. 21A) QRH*-KSP*-E3-IRDye800 (red), FIG. 21B) AF568-labeled anti-EGFR (yellow), and FIG. 21C) AF488-labeled anti-HER2 (green) show strong binding to the surface (arrows) of human OE33 xenograft tumor cells. FIG. 21D) Representative histology (H&E) shows boundary between normal and tumor. Heterodimer binding co-localizes with FIG. 21E) anti-EGFR and FIG. 21F) anti-HER2 with ρ=0.45 and 0.62, respectively. FIG. 21G) Quantified results show a significantly greater T/B ratio for the heterodimer (5.0±0.8) than either anti-EGFR (5.0±0.8) or anti-HER2 (3.2±0.8), P=3.4×10-13 and P=2.6×10-8 by unpaired t-test. Fluorescence intensities were quantified from a set of 3 boxes with dimensions of 20×20 μm2 placed randomly as shown in panels A-C). FIG. 21H-I) Western blot shows expression of EGFR and HER2 by OE33 and QhTERT cells.

FIG. 22A-C shows the peptide heterodimer: FIG. 22A) Schematic for labeling heterodimer with IRDye800. Experimental mass-to-charge (m/z) ratios for 22B) QRH*-KSP*-E3-SH and 22C) QRH*-KSP*-E3-IRDye800 were found to be 2385.29 and 3510.57, respectively, which were expected values.

FIG. 23A-F shows co-localization of heterodimer and antibody binding. Strong binding is seen by FIG. 23A) QRH*-KSP*-E3-IRDye800 (red), FIG. 23BB) AF568-labeled anti-EGFR antibody (yellow), and FIG. 23C) AF488-labeled anti-HER2 antibody (green) to the surface (arrow) of SKBR3 cells. Merged image of heterodimer with FIG. 23D) anti-EGFR and FIG. 23E) anti-HER2 result in Pearson's correlation coefficient of ρ=0.58 and 0.65, respectively. FIG. 23F) DAPI stain shows location of SKBr3 cell nuclei.

FIG. 24A-B shows immunohistochemistry for EGFR and HER2. Cell surface (arrow) expression of FIG. 24A) EGFR and FIG. 24B) HER2 is observed in OE33 tumor sections using immunohistochemistry.

### DESCRIPTION

Esophageal adenocarcinoma (EAC) is a heterogeneous disease that is rising rapidly in incidence and has a poor prognosis. In some embodiments, the disclosure provides a heterobivalent peptide ligand to target detection of early Barrett's neoplasia and/or Barrett's Esophagus (BE) by arranging monomer heptapeptides specific for either epidermal growth factor receptor (EGFR) or epidermal receptor growth factor 2 (ErbB2) in a heterodimer configuration comprising a triethyleneglycol linker that optimizes heterodimer binding to esophageal cells.

Progression of BE to EAC is characterized by high molecular heterogeneity that has limited the usefulness of conventional ligands that recognize a single target. In some embodiments, the disclosure provides a peptide heterodimer that binds both EGFR and ErbB2 concurrently for improved targeting performance. In some aspects, the heterobivalent peptide ligand, i.e., heterodimeric peptide, comprises an EGFR-specific peptide and an ErbB2-specific peptide, or a multimeric form of the heterodimeric peptide. In some aspects, the heterodimeric peptide comprises an EGFR-specific peptide QRHKPRE (SEQ ID NO: 1) and an ErbB2-specific peptide KSPNPRF (SEQ ID NO: 2), or a multimeric form of the heterodimeric peptide, wherein the heterodimeric peptide specifically binds to EGFR and ErbB2, and wherein at least one detectable label, or at least one therapeutic moiety, or both, are attached to the heterodimeric peptide or the multimeric form of the heterodimeric peptide.

As used herein, “EGFR” is epidermal growth factor receptor (also designated EGFR; ErbB-1, or HER1) is a transmembrane protein that is a receptor for members of the epidermal growth factor family (EGF family) of extracellular protein ligands.

As used herein, “ErbB2” is a receptor tyrosine-protein kinase (also known as CD340 (cluster of differentiation 340), proto-oncogene Neu, Erbb2 (rodent), ErbB2 (human), HER2 (from human epidermal growth factor receptor 2) or HER2/neu). ErbB-2 is alternatively called HER2 in humans and neu in rodents. ErbB is abbreviated from erythroblastic leukemia viral oncogene B, a gene isolated from avian genome.

The ErbB family of proteins contains four receptor tyrosine kinases, structurally related to EGFR, its first discovered member. In humans, the family includes Her1 (EGFR, ErbB1), Her2 (Neu, ErbB2), Her3 (ErbB3), and Her4 (ErbB4). Excessive ErbB signaling is associated with the development of a wide variety of types of solid tumor.

In some embodiments, the disclosure provides a reagent comprising a heterodimeric peptide comprising an EGFR-specific peptide and an ErbB2-specific peptide, or a multimeric form of the heterodimeric peptide, wherein the heterodimeric peptide specifically binds to EGFR and ErbB2, at least one detectable label. In some aspects, the EGFR-specific peptide is any peptide known to bind EGFR. In some aspects, the EGFR-specific peptide is any EGFR-specific peptide disclosed in international publication WO 2016/029125. In some aspects, the EGFR-specific peptide is QRHKPRE (SEQ ID NO: 1), HAHRSWS (SEQ ID NO: 9), YLTMPTP (SEQ ID NO: 10), TYPISFM (SEQ ID NO: 11), KLPGWSG (SEQ ID NO: 12), IQSPHFF (SEQ ID NO: 13), YSIPKSS (SEQ ID NO: 14), SHRNRPRNTQPS (SEQ ID NO: 15), NRHKPREKTFTD (SEQ ID NO: 5), TAVPLKRSSVTI (SEQ ID NO: 16), GHTANRQPWPND (SEQ ID NO: 17), LSLTRTRHRNTR (SEQ ID NO: 18), RHRDTQNHRPTN (SEQ ID NO: 19), ARHRPKLPYTHT (SEQ ID NO: 20), KRPRTRNKDERR (SEQ ID NO: 21), SPMPQLSTLLTR (SEQ ID NO: 22) or NHVHRMHATPAY (SEQ ID NO: 23). In some aspects, the ErbB2-specific peptide is any peptide known to bind ErbB2. In some aspects, the ErbB2-specific peptide is any ErbB2-specific peptide disclosed in international publication WO 2017/096036. In some aspects, the ERBB2-specific peptide is KSPNPRF (SEQ ID NO: 2), RHPFPRF (SEQ ID NO: 24), RHPWPNR (SEQ ID NO: 25), RHPYPQR (SEQ ID NO: 26) or RKPFPRH (SEQ ID NO: 27).

In some embodiments, the disclosure provides a reagent comprising a heterodimeric peptide comprising an EGFR-specific peptide QRHKPRE (SEQ ID NO: 1) and an ErbB2-specific peptide KSPNPRF (SEQ ID NO: 2), or a multimeric form of the heterodimeric peptide, wherein the heterodimeric peptide specifically binds to EGFR and ErbB2, at least one detectable label, i.e., Cy5.5, connected via an E3 linker, i.e., QRH*-KSP*-E3-Cy5.5.

In some embodiments, the heterobivalent peptide ligand (also designated heterodimeric peptide) or probe provides higher sensitivity and specificity for detection of Barrett's neoplasia in human esophageal specimens than with monoclonal antibodies to either target. No evidence of acute toxicity of the heterodimeric peptide was found on pathology. Thus, this heterodimeric binder provides a novel diagnostic and therapeutic tool in the diagnosis and treatment of esophageal disease.

By covalently linking two unique peptide sequences, multivalent ligand-target interactions are generated with improved binding affinity, providing a synergistic effect which is greater than the sum of that for the two individual monomers, allowing for targets to be detected at lower levels of expression. Higher sensitivity results from detection of two independent targets, and greater specificity arises from dual binding to a larger combined target epitope by comparison to the monomer [Yan et al, Amino Acids 41: 1081-92 (2011)].

Image-guided surgery that targets overexpression of molecules that are specific for EAC, high grade dysplasia (HGD) of the esophagus, or Barrett's neoplasia can help achieve a balance between complete tumor resection and maintenance of esophageal function. Targeted imaging can also help maximize the remaining volume of “normal” esophageal parenchyma to optimize post-operative function. In addition, imaging targets specific for EAC, HGD of the esophagus, or Barrett's neoplasia can serve as important biomarkers for evaluating patient prognosis.

The peptide heterodimeric reagent of the disclosure may be used as a diagnostic imaging agent to highlight foci of Barrett's neoplasia for guiding endoscopic mucosal resection (EMR). Imaging reagents can provide a biological basis for disease detection, prognosis, guide therapy, and monitor treatment response. Antibodies have been most commonly used, however they are large in size, high in molecular weight, and have long plasma half-lives, all leading to increased background on imaging. Peptides are attractive imaging tools, with a small size and low molecular weight that result in improved properties for deep tissue imaging inaccessible to antibodies. Peptides are less immunogenic, clear from non-target tissues to reduce background, and can be synthesized for improved binding affinity. All of this promotes deep tissue penetration and effective targeting.

In one aspect, the disclosure provides peptides that bind to EGFR and/or ErbB2 expressed on neoplastic cells, dysplastic cells, and/or cancerous cells. The peptides include, but are not limited to, the peptides QRHKPRE (SEQ ID NO: 1) and KSPNPRF (SEQ ID NO: 2), and a heterodimeric peptide comprising QRHKPRE (SEQ ID NO: 1) and KSPNPRF (SEQ ID NO: 2).

In a further aspect, the disclosure provides reagents comprising a peptide or heterodimeric peptide of the disclosure. A “peptide reagent” or “heterodimeric peptide” of the disclosure comprises at least two components, a peptide or heterodimeric peptide and another moiety attached to the peptide or heterodimeric peptide. The only component of the reagent that contributes to binding of EGFR and/or ErbB2 is the heterodimeric peptide of the disclosure. In other words, the reagent “consists essentially of” a peptide or heterodimeric peptide of the disclosure. In some embodiments, the other moiety comprises amino acids but the peptide or heterodimeric peptide of the disclosure is not linked to those amino acids in nature and the other amino acids do not affect binding of the peptide or heterodimeric peptide to EGFR and/or ErbB2. Moreover, the other moiety in a reagent contemplated herein is not a phage in a phage display library or a component of any other type of peptide display library.

In some embodiments, the reagents comprise at least one detectable label as a moiety attached to a peptide of the disclosure. The detectable label, in some aspects is detected, for example, by microscopy, ultrasound, PET, SPECT, or magnetic resonance imaging. In some aspects, the label detectable by microscopy is fluorescein isothiocyanate (FITC), Cy5, Cy5.5 and IRdye800. In exemplary aspects, a high T/B ratio was achieved using Cy5.5 to emit NIR fluorescence, which is least affected by hemoglobin absorption, tissue scattering, and autofluorescence background [Becker et al., Nat. Biotechnol. 19: 327-31 (2001)], and provided maximum tumor imaging depth.

In some embodiments, the detectable label is attached to a peptide or heterodimeric peptide of the disclosure by one or more linkers. In some aspects, a linker is attached at the end of each peptide monomer in the heterodimer. In some aspects, the linker(s) separates the peptide or heterodimeric peptide and the fluorophore to prevent steric hindrance. The linker, in various aspects, is connected at the N- or C-terminus of the peptide. In particular aspects, the linker is attached at the C-terminus of each peptide monomer. In some aspects, the linker(s) is an aminohexonic acid. In some aspects, the linker(s) is a polyethylene glycol (PEG) or a polyethylene oxide (PEO), or derivatives thereof. In some aspects, the linker is PEG2, PEG3, PEG6, PEG10, or a PEG phosphate. In some aspects, the linker is triethylene glycol (PEG3 or E3). In some aspects, the linker comprises a length of about 60 Å. In some aspects, a linker length of about 60 Å is needed to conform to the spacing between domains 2 of EGFR and 3 of ErbB2.

In some aspects, the linker is a sequence of amino acids located at the C-terminus of a peptide or heterodimeric peptide. In some aspects, the linker is connected to the peptide by a solid phase peptide synthesizer. In some aspects, the linker sequence terminates with a lysine residue. Thus, in some aspects, the linker is a peptide. In some aspects, the linker sequence is GGGSK (SEQ ID NO: 3). In some aspects, the linker sequence is GGGAGGG (SEQ ID NO: 28). In some aspects, the linker is a branched peptide sequence. In some aspects, one peptide sequence, e.g., GGGAGGG (SEQ ID NO: 28) is connected to each peptide binder in the heterodimeric peptide. In some aspects, a Lysine (K) residue is connected at the C-terminus, of each peptide sequence, e.g., GGGAGGGK (SEQ ID NO: 29), and the two lysines at the end of each linker are connected to each other via a peptide bond. Therefore, in some aspects, such a linker may be considered to be GGGAGGGK (SEQ ID NO: 29).

In some aspects, the monomeric peptides, which bind EGFR and ErbB2, are each connected to a linker which is attached to a detectable label. In some aspects, the monomeric peptides, which bind EGFR and ErbB2, are attached to the linker at their C-terminus or their N-terminus. In some aspects, the monomeric peptides are provided in either orientation. In some aspects, the linker is connected at the C-terminus of the peptide.

In some exemplary aspects, the linker is an E3 linker connected at the C-terminus of each of the monomeric peptides. In some aspects, the E3 linkers are further connected via one or more lysines to the detectable label in the following manner.

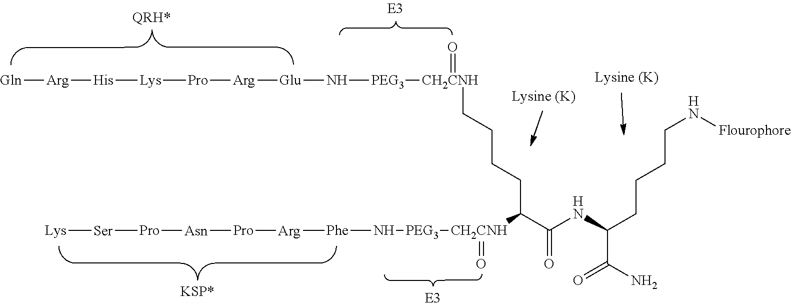

In some embodiments, the reagents comprise at least one therapeutic moiety attached to peptide or heterodimeric peptide of the disclosure. In some aspects, the therapeutic moiety is a chemopreventative or chemotherapeutic agent. In some aspects, the chemotherapeutic moiety is celecoxib, 5-fluorouracil (5-FU), and/or chlorambucil. In some aspects, the chemotherapeutice moieties are carboplatin and paclitaxel; cisplatin and 5-fluorouracil (5-FU); ECF: epirubicin (Ellence®), cisplatin, and 5-FU; DCF: docetaxel (Taxotere®), cisplatin, and 5-FU; cisplatin with capecitabine; oxaliplatin and either 5-FU or capecitabine; or irinotecan. In some aspects, the therapeutic moiety is a micelle encapsulating a therapeutic moiety. In certain embodiments, the micelle encapsulates carboplatin and paclitaxel; cisplatin and 5-fluorouracil (5-FU); ECF: epirubicin (Ellence®), cisplatin, and 5-FU; DCF: docetaxel (Taxotere®), cisplatin, and 5-FU; cisplatin with capecitabine; oxaliplatin and either 5-FU or capecitabine; or irinotecan. In some embodiments, the micelle comprises trastuzumab or ramucirumab.

In some embodiments, the reagent comprises at least one detectable label attached to the peptide or heterodimeric peptide, or multimeric form of the peptide or heterodimeric peptide. In some embodiments, the reagent comprises at least one therapeutic moiety attached to the peptide or heterodimeric peptide, or multimeric form of the peptide or heterodimeric peptide. In some embodiments, the reagent comprises at least one detectable label and at least one therapeutic moiety attached to the peptide or heterodimeric peptide, or multimeric form of the peptide or heterodimeric peptide.

In yet a further aspect, the disclosure provides a composition comprising a reagent of the disclosure and a pharmaceutically acceptable excipient.

In still a further aspect, the disclosure provides a method for specifically detecting EAC, HGD of the esophagus, or Barrett's neoplasia in a patient comprising the steps of administering a reagent of the disclosure attached to a detectable label to the patient and detecting binding of the reagent to EAC cells, HGD cells of the esophagus, and/or Barrett's neoplastic cells. In some embodiments, the detectable binding takes place in vivo. In others, the detectable binding takes places in vitro. In still others, the detectable binding takes place in situ.

The phrase “specifically detects” means that the reagent binds to and is detected in association with a type of cell, and the reagent does not bind to and is not detected in association with another type of cell at the level of sensitivity at which the method is carried out.

In an additional aspect, the disclosure provides a method of determining the effectiveness of a treatment for EAC, HGD of the esophagus, Barrett's neoplasia, cancer metastasis, or recurrence of cancer in a patient comprising the step of administering a reagent of the disclosure attached to a detectable label to the patient, visualizing a first amount of cells labeled with the reagent, and comparing the first amount to a previously-visualized second amount of cells labeled with the reagent, wherein a decrease in the first amount cells labeled relative to the previously-visualized second amount of cells labeled is indicative of effective treatment. In some embodiments, a decrease of 5% is indicative of effective treatment. In other embodiments, a decrease of about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more is indicative of effective treatment. In some embodiments, the method further comprises obtaining a biopsy of the cells labeled by the reagent.

In another aspect, the disclosure provides a method for delivering a therapeutic moiety to a patient comprising the step of administering a reagent of the disclosure attached to a therapeutic moiety to the patient.

In yet another aspect, the disclosure provides a method for delivering a therapeutic moiety to EAC cells, HGD cells of the esophagus, or Barrett's neoplastic cells of a patient comprising the step of administering a reagent of the disclosure attached to a therapeutic moiety to the esophagus of the patient.

In still another aspect, the disclosure provides a kit for administering a composition of the disclosure to a patient in need thereof, where the kit comprises a composition of disclosure, instructions for use of the composition and a device for administering the composition to the patient.

### Linkers, Peptides and Peptide Analogs

In some embodiments, two monomers are combined in a heterodimer configuration using a variable linker designed with either a PEG spacer that ranged in size from 17-74 atoms or a hydrophobic 6-aminohexonic acid linker in the α-, ξ-amino groups of the second lysine. Linker length is varied to determine the optimal spacing between monomers to maximize binding of the heterodimeric peptide to the intended targets. A key step in optimizing signal is matching the length of the linkers in the heterodimer with the mean distance between the extracellular binding domains of either target.

In some embodiments, the detectable label is attached to a peptide or heterodimeric peptide of the disclosure by one or more linkers. In some embodiments, the detectable label is attached to a peptide or heterodimeric peptide of the disclosure by one or more linkers. In some aspects, a linker is used to connect the different monomers in the bivalent heterodimeric peptide. In some aspects, a linker is used to connect the peptide or heterodimeric peptide to a fluorophore or therapeutic moiety. In some aspects, a linker is attached at the end of each peptide monomer in the heterodimer. In some aspects, the linker(s) separates the peptide or heterodimeric peptide and the fluorophore to prevent steric hindrance. The linker, in various aspects, is connected at the N- or C-terminus of the peptide. In particular aspects, the linker is attached at the C-terminus of each peptide monomer. In some aspects, the linker(s) is an aminohexonic acid. In some aspects, the linker(s) is a polyethylene glycol (PEG) or a polyethylene oxide (PEO), or derivatives thereof. In some aspects, the linker is PEG2, PEG3, PEG6, PEG10, or a PEG phosphate. In some aspects, the linker is triethylene glycol (PEG3 or E3). In some aspects, the linker comprises a length of about 60 Å. In some aspects, a linker length of about 60 Å is needed to conform to the spacing between domains 2 of EGFR and 3 of ErbB2.

In some aspects, the linker is a sequence of amino acids located at the C-terminus of a peptide or heterodimeric peptide. In some aspects, the linker is connected to the peptide by a solid phase peptide synthesizer. In some aspects, the linker sequence terminates with a lysine residue. Thus, in some aspects, the linker is a peptide. In some aspects, the linker sequence is GGGSK (SEQ ID NO: 3). In some aspects, the linker sequence is GGGAGGG (SEQ ID NO: 28). In some aspects, the linker is a branched peptide sequence. In some aspects, one peptide sequence, e.g., GGGAGGG (SEQ ID NO: 28) is connected to each peptide binder in the heterodimeric peptide. In some aspects, a Lysine (K) residue is connected at the C-terminus of each peptide sequence, e.g., GGGAGGGK (SEQ ID NO: 29), and the two lysines at the end of each linker are connected to each other via a peptide bond. Therefore, in some aspects, such a linker also is considered to be GGGAGGGK (SEQ ID NO: 29).

In some aspects, the monomeric peptides, which bind EGFR and ErbB2, are each connected to a linker which is attached to a detectable label. In some aspects, the monomeric peptides, which bind EGFR and ErbB2, are attached to the linker at their C-terminus or their N-terminus. In some aspects, the monomeric peptides are provided in either orientation. In some aspects, the linker is connected at the C-terminus of the peptide.

In some exemplary aspects, the linker is an E3 linker connected at the C-terminus of each of the monomeric peptides. In some aspects, the E3 linkers are further connected via one or more lysines to the detectable label in the following manner.

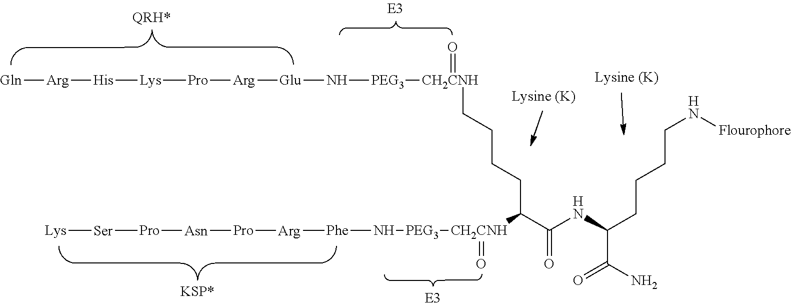

In some embodiments, the presence of a linker results in at least a 1% increase in detectable binding of a reagent of the disclosure to the esophageal cells including, but not limited to OE33 and QhTERT esophageal cells, Barrett's esophageal cells, high grade dysplastic (HGD) esophageal cells, esophageal adenocarcinoma cells (EAC), and esophageal squamous carcinoma cells, compared to the detectable binding of the reagent in the absence of the linker. In various aspects, the increase in detectable binding is at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 100-fold or more.

The term “peptide” refers to molecules of 2 to 50 amino acids, molecules of 3 to 20 amino acids, and those of 6 to 15 amino acids. Peptides and linkers as contemplated by the disclosure may be 5 amino acids in length. In various aspects, a polypeptide or linker may be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length.

The term “heterodimeric peptide” is used interchangeably with the term “heterobivalent peptide” and refers to a molecule comprising two monomer peptides designed to bind or detect two different targets. In exemplary aspects, the heterodimeric peptide or heterobivalent peptide binds both EGFR and ErbB2. In exemplary aspects, the heterodimeric peptide comprises the heptapeptide monomers QRHKPRE (QRH*) and KSPNPRF (KSP*). In some aspects, the heptapeptide monomers QRHKPRE (QRH*) and KSPNPRF (KSP*) are covalently linked. In some aspects, the heptapeptide monomers QRHKPRE (QRH*) and KSPNPRF (KSP*) are joined by a linker. In some aspects, a linker is joined to each peptide monomer. In some aspects, a linker is joined to the N-terminus or the C-terminus of each peptide monomer. In more particular aspects, a linker is joined to the C-terminus of each peptide monomer of the heterodimeric peptide. In some aspects, the linker is any linker known to one of ordinary skill which maintains the heterodimer in the proper configuration to bind to its targets and to a detectable label. In some aspects, the linker is any of the linkers described herein throughout the disclosure. In further exemplary aspects, the heterodimeric peptide is joined with an E3 linker and a label, e.g., QRH*-KSP*-E3-Cy5.5.

Exemplary peptides used in heterodimeric peptides of the disclosure are, in various aspects, randomly generated by methods known in the art, carried in a polypeptide library (for example and without limitation, a phage display library), derived by digestion of proteins, or chemically synthesized. Peptides exemplified in the disclosure have been developed using techniques of phage display, a powerful combinatorial method that uses recombinant DNA technology to generate a complex library of polypeptides for selection by preferential binding to cell surface targets [Scott et al., Science, 249:386-390 (1990)]. The protein coat of bacteriophage, such as the filamentous M13 or icosahedral T7, is genetically engineered to express a very large number (>10⁹) of different polypeptides with unique sequences to achieve affinity binding [Cwirla et al., Proc. Natl. Acad. Sci. USA, 87:6378-6382 (1990)]. Selection is then performed by biopanning the phage library against cultured cells and tissues that over express the target. The DNA sequences of these candidate phage are then recovered and used to synthesize the polypeptide [Pasqualini et al., Nature, 380:364-366 (1996)]. The polypeptides that preferentially bind to GPC3 are optionally labeled with fluorescence dyes, including but not limited to, FITC, Cy 5.5, Cy 7, and Li-Cor.

Peptides include D and L forms, either purified or in a mixture of the two forms. Also contemplated by the disclosure are peptides that compete with peptides of the disclosure for binding to OE33 and QhTERT esophageal cells, Barrett's esophageal cells, high grade dysplastic (HGD) esophageal cells, esophageal adenocarcinoma cells (EAC), and/or esophageal squamous carcinoma cells.

In some embodiments, a peptide or heterodimeric peptide of a reagent of the disclosure is presented in multimeric form. In some aspects, the heterodimeric peptides can aggregate to form further multimers. Various scaffolds are known in the art upon which multiple peptides can be presented. In some embodiments, a peptide is presented in multimer form on a trilysine dendritic wedge. In some embodiments, a peptide is presented in dimer form using an aminohexanoic acid linker. Other scaffolds known in the art include, but are not limited to, other dendrimers and polymeric (e.g., PEG) scaffolds.

It will be understood that peptides, heterodimeric peptides, and linkers of the disclosure optionally incorporate modifications known in the art and that the location and number of such modifications are varied to achieve an optimal effect in the peptide and/or linker analog.

In some embodiments, the compound is a peptide analog having a structure based on one of the peptides disclosed herein (the “parent peptide”) but differs from the parent peptide in one or more respects. Accordingly, as appreciated by one of ordinary skill in the art, the teachings regarding the parent peptides provided herein may also be applicable to the peptide analogs.

In some embodiments, the peptide analog comprises the structure of a parent peptide, except that the peptide analog comprises one or more non-peptide bonds in place of peptide bond(s). In some embodiments, the peptide analog comprises in place of a peptide bond, an ester bond, an ether bond, a thioether bond, an amide bond, and the like. In some embodiments, the peptide analog is a depsipeptide comprising an ester linkage in place of a peptide bond.

In some embodiments, the peptide analog comprises the structure of a parent peptide described herein, except that the peptide analog comprises one or more amino acid substitutions, e.g., one or more conservative amino acid substitutions. Conservative amino acid substitutions are known in the art, and include amino acid substitutions in which one amino acid having certain physical and/or chemical properties is exchanged for another amino acid that has the same chemical or physical properties. For instance, the conservative ammo acid substitution may be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

In some aspects, the peptide analog comprises one or more synthetic amino acids, e.g., an amino acid non-native to a mammal. Synthetic amino acids include β-alanine (β-Ala), N-α-methyl-alanine (Me-Ala), aminobutyric acid (Abu), γ-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (MeIle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O₂)), norleucine (Nle), methyl-norleucine (Me-Nle), norvaline (Nva), ornithine (Om), para-aminobenzoic acid (PABA), penicillamine (Pen), methylphenylalanine (MePhe), 4-Chlorophenylalanine (Phe(4-Cl)), 4-fluorophenylalanine (Phe(4-F)), 4-nitrophenylalanine (Phe(4-NO₂)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), O-benzyl-phosphotyrosine, β-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid.

In some embodiments, the peptide analog comprises one or more non-conservative amino acid substitutions and the peptide analog still functions to a similar extent, the same extent, or an improved extent as the parent peptide. In certain embodiments, the peptide analog comprising one or more non-conservative amino acid substitutions exhibits about the same or greater binding to OE33 and QhTERT esophageal cells, Barrett's esophageal cells, high grade dysplastic (HGD) esophageal cells, esophageal adenocarcinoma cells (EAC), and/or esophageal squamous carcinoma cells in comparison to the parent peptide.

In some embodiments, the peptide analog comprises one or more amino acid insertions or deletions, in comparison to the parent peptide described herein. In some embodiments, the peptide analog comprises an insertion of one or more amino acids in comparison to the parent peptide. In some embodiments, the peptide analog comprises a deletion of one or more amino acids in comparison to the parent peptide. In some embodiments, the peptide analog comprises an insertion of one or more amino acids at the N- or C-terminus in comparison to the parent peptide. In some embodiments, the peptide analog comprises a deletion of one or more amino acids at the N- or C-terminus in comparison to the parent peptide. In these embodiments, the peptide analog still exhibits about the same or greater binding to HCC cells in comparison to the parent peptide.

### Detectable Markers

As used herein, a “detectable marker” is any label that can be used to identify the binding of a composition or reagent of the disclosure to OE33 and QhTERT esophageal cells, Barrett's esophageal cells, high grade dysplastic (HGD) esophageal cells, esophageal adenocarcinoma cells (EAC), and/or esophageal squamous carcinoma cells. Non-limiting examples of detectable markers are fluorophores, chemical or protein tags that enable the visualization of a polypeptide. Visualization in certain aspects is carried out with the naked eye, or a device (for example and without limitation, an endoscope) and may also involve an alternate light or energy source.

Fluorophores, chemical and protein tags that are contemplated for use in the disclosure include, but are not limited to, FITC, Cy 5, Cy 5.5, Cy 7, Li-Cor, a radiolabel, biotin, luciferase, 1,8-ANS (1-Anilinonaphthalene-8-sulfonic acid), 1-Anilinonaphthalene-8-sulfonic acid (1,8-ANS), 5-(and-6)-Carboxy-2′, 7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-γ-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, γ-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, C5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, DiI, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, eYFP (Enhanced Yellow Fluorescent Protein), FDA, FITC antibody conjugate pH 8.0, FlAsH, Fluo-3, Fluo-3 Ca2⁺, Fluo-4, Fluor-Ruby, Fluorescein, Fluorescein 0.1 μM NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2⁺, Fura Red, high Ca, Fura Red, low Ca, Fura-2 Ca2+, Fura-2, Fura-2, GFP (S65T), HcRed, Indo-1 Ca2⁺, Indo-1, Ca free, Indo-1, Ca saturated, JC-1, JC-1 pH 8.2, Lissamine rhodamine, Lucifer Yellow, CH, Magnesium Green, Magnesium Green Mg2⁺, Magnesium Orange, Marina Blue, mBanana, mCherry, mHoneydew, mOrange, mPlum, mRFP, mStrawberry, mTangerine, NBD-X, NBD-X, MeOH, NeuroTrace 500/525, green fluorescent Nissl stain-RNA, Nile Blue, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, R-Phycoerythrin pH 7.5, ReAsH, Resorufin, Resorufin pH 9.0, Rhod-2, Rhod-2 Ca2⁺, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodamine Green pH 7.0, Rhodol Green antibody conjugate pH 8.0, Sapphire, SBFI-Na+, Sodium Green Na⁺, Sulforhodamine 101, Tetramethylrhodamine antibody conjugate pH 8.0, Tetramethylrhodamine dextran pH 7.0, and Texas Red-X antibody conjugate pH 7.2.

Non-limiting examples of chemical tags contemplated by the disclosure include radiolabels. For example and without limitation, radiolabels that contemplated in the compositions and methods of the disclosure include ¹¹C, ¹³N, ¹⁵O, ¹⁸F, ³²P, ⁵²Fe, ⁶²Cu, ⁶⁴Cu, ⁶⁷Cu, ⁶⁷Ga, ⁶⁸Ga, ⁸⁶Y, ⁸⁹Z, ⁹⁰Y, ⁹⁴mTc, ⁹⁴Tc, ⁹⁵Tc, ⁹⁹mTc, ¹⁰³Pd, ¹⁰⁵Rh, ¹⁰⁹Pd, ¹¹¹Ag, ¹¹¹In, ¹²³I, ¹²⁴I, ¹²⁵I, ¹³¹I, ¹⁴⁰La, ¹⁴⁹Pm, ¹⁵³Sm, ¹⁵⁴⁻¹⁵⁹Gd, ¹⁶⁵Dy, ¹⁶⁶Dy, ¹⁶⁶Ho, ¹⁶⁹Yb, ¹⁷⁵Yb, ¹⁷⁵Lu, ¹⁷⁷Lu, ¹⁸⁶Re, ¹⁸⁸Re, ¹⁹²Ir, ¹⁹⁸Au, ¹⁹⁹Au, and ²¹²Bi.

A worker of ordinary skill in the art will appreciate that there are many such detectable markers that can be used to visualize a cell, in vitro, in vivo or ex vivo. In exemplary aspects, the detectable marker is fluorescein isothiocyanate (FITC), Cyanine 5 (Cy5), Cyanine 5.5 (Cy5.5), and/or near-infrared (NIR) fluorescent dye 800 (IRDye800).

### Therapeutic Moieties

Therapeutic moieties contemplated by the disclosure include, but are not limited to polypeptides (including protein therapeutics) or peptides, small molecules, chemotherapeutic agents, or combinations thereof.

The term “small molecule”, as used herein, refers to a chemical compound, for instance a peptidometic or oligonucleotide that may optionally be derivatized, or any other low molecular weight organic compound, either natural or synthetic.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

In some embodiments, the therapeutic moiety is a protein therapeutic. Protein therapeutics include, without limitation, cellular or circulating proteins as well as fragments and derivatives thereof. Still other therapeutic moieties include polynucleotides, including without limitation, protein coding polynucleotides, polynucleotides encoding regulatory polynucleotides, and/or polynucleotides which are regulatory in themselves. Optionally, the compositions comprise a combination of the compounds described herein.

In some embodiments, protein therapeutics include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2a, cytokine-induced neutrophil chemotactic factor 2β, β, endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor 3 binding protein I, transforming growth factor 3 binding protein II, transforming growth factor 3 binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Therapeutic moieties also include, in some embodiments, chemotherapeutic agents. A chemotherapeutic agent contemplated for use in a reagent of the disclosure includes, without limitation, alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens, such as flutamide. Chemotherapeutic agents such as gefitinib, sorafenib and erlotinib are also specifically contemplated.

In more particular embodiments, some common drugs and drug combinations used in the treatment of esophageal cancer include, but are not limited to, carboplatin and paclitaxel (TAXOL®) (which may be combined with radiation); cisplatin and 5-fluorouracil (5-FU) (often combined with radiation); ECF: epirubicin (ELLENCE®), cisplatin, and 5-FU (especially for gastroesophageal junction tumors); DCF: docetaxel (TAXOTERE®), cisplatin, and 5-FU; cisplatin with capecitabine (XELODA®); oxaliplatin and either 5-FU or capecitabine; and irinotecan (Captosar). For some esophagus cancers, chemotherapy may be used along with the targeted drug trastuzumab (HERCEPTIN®) or ramucirumab (Cyramza).

Therapeutic moieties also include micelles that, in turn, encapsulate another therapeutic moiety. In some embodiments, the micelles are polymeric micelles such as octadecyl lithocholate micelles. Peptides or heterodimeric peptides described herein are attached to polymeric micelles such as octadecyl lithocholate micelles described in Khondee et al., J. Controlled Release 199: 114-121 (2015) and U.S. Provisional Patent Application No. 62/262,195. In some embodiments, the micelles encapsulate a chemotherapeutice drug including, but not limited to, carboplatin and paclitaxel (TAXOL®) (which may be combined with radiation); cisplatin and 5-fluorouracil (5-FU) (often combined with radiation); ECF: epirubicin (ELLENCE®), cisplatin, and 5-FU (especially for gastroesophageal junction tumors); DCF: docetaxel (TAXOTERE®), cisplatin, and 5-FU; cisplatin with capecitabine (XELODA®); oxaliplatin and either 5-FU or capecitabine; and irinotecan (Captosar). For some esophagus cancers, chemotherapy may be used along with the targeted drug trastuzumab (HERCEPTIN®) or ramucirumab (Cyramza).

Dosages of the therapeutic moiety provided are administered as a dose measured in, for example, mg/kg. Contemplated mg/kg doses of the disclosed therapeutics include about 1 mg/kg to about 60 mg/kg. Specific ranges of doses in mg/kg include about 1 mg/kg to about 20 mg/kg, about 5 mg/kg to about 20 mg/kg, about 10 mg/kg to about 20 mg/kg, about 25 mg/kg to about 50 mg/kg, and about 30 mg/kg to about 60 mg/kg. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

“Effective amount” as used herein refers to an amount of a reagent of the disclosure sufficient to visualize the identified disease or condition, or to exhibit a detectable therapeutic or inhibitory effect. The effect is detected by, for example, an improvement in clinical condition or reduction in symptoms. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

### Visualization of Reagents

Visualization of binding to any of the cells described herein in the disclosure is by any means known to those of ordinary skill in the art. As discussed herein, visualization is, for example and without limitation, in vivo, in vitro, ex vivo, or in situ visualization.

In some embodiments where the detectable label is a radiolabel, the radiolabel is detected by nuclear imaging.

In some embodiments where the detectable label is a fluorophore, the fluorophore is detected by near infrared (NIR) fluorescence imaging.

Some embodiments of methods of the disclosure involve the acquisition of a tissue sample from a patient. The tissue sample is selected from the group consisting of a tissue or organ of said patient.

### Formulations

Compositions of the disclosure are formulated with pharmaceutically acceptable excipients such as carriers, solvents, stabilizers, adjuvants, diluents, etc., depending upon the particular mode of administration and dosage form. The compositions are generally formulated to achieve a physiologically compatible pH, and range from a pH of about 3 to a pH of about 11, about pH 3 to about pH 7, depending on the formulation and route of administration. In alternative embodiments, the pH is adjusted to a range from about pH 5.0 to about pH 8. In various aspects, the compositions comprise a therapeutically effective amount of at least one compound as described herein, together with one or more pharmaceutically acceptable excipients. Optionally, the compositions comprises a combination of the compounds described herein, or may include a second active ingredient useful in the treatment or prevention of bacterial growth (for example and without limitation, anti-bacterial or anti-microbial agents), or may include a combination of reagents of the disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol) wetting or emulsifying agents, pH buffering substances, and the like.

### Kits

In some embodiments, the disclosure includes kits. In various aspects, the kit comprises reagents of the disclosure, including the heterodimeric peptides described herein. In some aspects, the kit contains a label that describes use of the reagents provided in the kit. In some aspects, the label includes instruction for use. In some aspects, the kit further comprises a device for administering the composition or the heterodimeric peptide to a patient or to the cells of a patient for detecting, targeting, diagnosing and/or treating diseases. Such diseases include various diseases and cancers of the esophagus as described herein.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

All publications, patents and patent applications cited in this specification are herein incorporated by reference as if each individual publication or patent application were specifically and individually indicated to be incorporated by reference in its entirety to the extent that it is not inconsistent with the disclosure.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The disclosure will be more fully understood by reference to the following examples which detail exemplary embodiments of the disclosure.

### Example 1

Peptides that Bind EGFR

Peptides that bind specifically to EGFR were made as previously described in International Patent Application No. PCT/US2015/046314 (published international application number WO 2016/029125). Peptide selection was performed per manufacturer instructions using a phage display library (New England Biolabs) that consists of M13 bacteriophage that expresses ˜10⁹ unique 12-amino acid sequences. The phage library (2×10¹¹ pfu consisting of 2×10⁹ unique clones with ˜100 copies each) was biopanned against the purified recombinant EGFR-ECD proteins immobilized in a 6-well plate at 4° C. The biopanning was performed with the known protein target (EGFR-ECD) as opposed to the unbiased approach performed in Li et al., Gastroenterology, 139:1472-80 (2010). Four rounds of biopanning were performed using a decreasing quantity (100, 80, 60, and 40 μg) of EGFR-ECD in successive rounds to increase binding specificity. After four rounds of biopanning, fifty phage colonies were randomly selected for DNA preparation and sequencing analysis. These sequences were analyzed with an ABI Automatic DNA Analyzer (Applied Biosystems) using the primer 5′-CCCTCATAGTTAGCGTAACG-3′ (SEQ ID NO: 8)(−96 gIII sequencing primer, New England Biolabs) that corresponds to the pIII gene sequence of the M13 phage.

PepSite software [Petsalaki et al., PLoS Comput Biol. 5:e1000335 (2009)] was used to evaluate binding of the candidate sequences to the crystal structure of the extra-cellular domain of inactive, monomeric EGFR (code: 1IVO) obtained from the RCSB Protein Data Bank. 3D biochemical structures of the peptides were created using Chembiodraw software (Perkin Elmer). To achieve the highest specificity, the peptide sequences obtained from biopanning were mutated to align with the pdb structure of EGFR extracellular domain. For example, the first seven amino acids of the candidate sequence NRHKPREKTFTD (SEQ ID NO: 5) were chosen, and mutated first N to Q to generate the peptide sequence QRHKPRE (SEQ ID NO: 1). Alignment of Cy5.5-labeled peptides was validated on a structural model (Hex 6.3, Inria) [Macindoe et al., Nucleic Acids Research 38(S2):W445-W449 (2010)] by rotating the receptor and ligand about their centers of mass over a full range of intermolecular distances and rotational angles.

Using the structural model described in Petsalaki et al., supra, a minimum docking energy of Eₜ=−504.1 was identified for QRHKPRE (SEQ ID NO: 1) labeled with Cy5.5 to EGFR (1IVO). This sequence was synthesized and the Cy5.5 fluorophore was attached via a GGGSK (SEQ ID NO: 3) linker on the C-terminus, hereafter QRH*-Cy5.5. Cy5.5 was chosen for its high quantum yield and photostability. The linker was used to prevent steric hindrance. A scrambled sequence PEHKRRQ (SEQ ID NO: 6) was developed for use as a control, hereafter PEH*-Cy5.5, and found Eₜ=−493.1. On the model, QRH*-Cy5.5 binds to amino acids 230-310 of domain 2 of EGFR. Human and mouse EGFR have 97.5% homology in this region. The fluorescence spectra of QRH*-Cy5.5 and PEH*-Cy5.5 at 10 μM concentration in PBS with, =671 nm excitation revealed a peak emission at 710 nm. The Cy5.5-labeled peptides were purified to >97% on HPLC, and measured an experimental mass-to-charge (m/z) ratio on mass spectrometry of 1900.05 for both QRH*-Cy5.5 and PEH*-Cy5.5, which agreed with expected values.

Cy5.5-labeled peptides were synthesized using standard Fmoc-mediated solid-phase synthesis [Fields et al., Int. J. Pept. Protein Res. 35: 161-214 (1990)]. Fmoc and Boc protected L-amino acids were used, and synthesis was assembled on rink amide MBHA resin. The peptides were synthesized on a PS3 automatic synthesizer (Protein Technologies Inc). The C-terminal lysine was incorporated as Fmoc-Lys (ivDde)-OH, and the N-terminal amino acid was incorporated with Boc protection to avoid unwanted Fmoc removal during deprotection of the ivDde moiety prior to fluorophore labeling. Upon complete assembly of the peptide, the resin was transferred to a reaction vessel for manual labeling with dye. The ivDde side chain protecting group was removed with 5% hydrazine in DMF (3×10 min) with continuous shaking at room temperature (RT). The resin was washed with Dimethylformamide (DMF) and dichloromethane (DCM) 3× each for 1 min. The protected resin-bound peptide was incubated overnight with Cy5.5-NHS ester (Lumiprobe LLC) with DIEA, and the completion of the reaction was monitored by a qualitative Ninhydrin test. Upon completion of labeling, the peptide was cleaved from the resin using TFA: TIS: H2O (95:2.5:2.5 v/v/v; Sigma-Aldrich) for 4 hours with shaking in the dark at RT. After separation of the peptide from the resin, the filtrate was evaporated with N₂ gas followed by precipitation with chilled diethyl ether and stored overnight at −20° C. The precipitate was centrifuged at 3000 rpm for 5 min and washed with diethyl ether 3× and centrifuged in between each washing step. The crude peptides were dissolved in 1:1 Acetonitrile/H2O (v/v) and purified by prep-HPLC with a C18 column (Waters Inc) using a water (0.1% TFA)-acetonitrile (0.1% TFA) gradient. The final purity of the peptides was confirmed by analytical C18-column. Further characterization was performed with either ESI (Waters Inc) or Q-TOF (Agilent Technologies) mass spectrometry.

### Example 2

Peptides that Bind HER2 (ErbB2)

Peptides that bind specifically to the extra-cellular domain (ECD) of HER2 (2A91) were made as previously described in International Patent Application No. PCT/US16/64410 (published international application number WO 2017/096036) (using a structural model [Garrett et al., Mol. Cell. 11: 495-505 (2003)]. The ECD was targeted because it is accessible to imaging. About 1000 candidates were generated using mimotopes by considering Arg or Lys amino acid residues at the N-terminus to form hydrophilic interactions with HER2-ECD [Lemmon, Exp. Cell Res., 315: 638-648 (2009); Franklin et al., Cancer Cell, 5: 317-28 (2009)]. Hydrophobic residues, such as Phe, Trp, Val, Met, Ile, and Leu, were appended at the C-terminus to increase the likelihood of hydrophobic/hydrophilic interaction (Garrett et al., supra). Other amino acids, such as Ser, His, Arg, Tyr, Thr, Asp, and Asn, were used to increase peptide diversity [Wang et al., Anal Chem. 18: 8367-72 (2015)]. Either the N- or C-terminus of the peptides was connected by a conformationally rigid spacer group, such as PFP, PNP, PYP, and PWP, in the middle region. The ability of the candidates to bind to HER2-ECD domain 1-3 was evaluated using Pepsite-2 [Trabuco et al., Nucleic Acids Res., 40: W423-26 (2012)]. Five leading candidates, based on β-value, were evaluated, and KSPNPRF (SEQ ID NO: 2) was selected.

A FITC label was attached to each peptide, and binding to Flo1-HER2 and QhTERT cells (control) was imaged using confocal microscopy. Western blots of these cells showed differences in HER2 expression. KSPNPRF (SEQ ID NO: 2) was selected based on the highest mean fluorescence intensity with binding to Flo1-HER2. From the model, this peptide was determined to bind to HER2 domain 3. The peptide was then labeled for deep tissue imaging with a Cy5.5 fluorophore via a GGGSK (SEQ ID NO: 3) linker on the C-terminus, hereafter KSP*-Cy5.5. Cy5.5 was chosen for high quantum yield and photostability. The linker prevents steric hindrance by spatially separating the fluorophore from the peptide. A scrambled sequence PPSNFKR (SEQ ID NO: 4) was developed for use as control by altering the conformationally rigid spacer PNP and moving both hydrophobic and hydrophilic amino acids at the C-terminus. This control peptide also was linked to Cy5.5 via GGGSK (SEQ ID NO: 3), hereafter PPS*-Cy5.5. The absorbance spectra of KSP*-Cy5.5 and PPS*-Cy5.5 at 5 μM in PBS showed a maximum at 680 nm. The fluorescence emission peak occurs at 708 nm in the NIR spectrum. For both peptides, >98% purity was achieved with HPLC, and measured an experimental mass-to-charge (m/z) ratio on mass spectrometry of 1794.98, which agreed with expected values.

### Example 3

### Cells, Media, and Chemicals

All cells in the experiments of the disclosure, unless otherwise indicated, were maintained at 37° C. and 5% CO₂ and were supplemented with 10% FBS and 1% penicillin/streptomycin. Penicillin/streptomycin was omitted for the siRNA knockdown studies, and FBS was omitted in keratinocyte-SFE media. Human SKBr3 breast cancer cells were obtained from the American Type Culture Collection (ATCC, Manassas, VA) and cultured in McCoy's 5 Å media. A human esophageal adenocarcinoma cell line (OE33) and a non-dysplastic Barrett's esophageal cell line (QhTERT) were cultured with Roswell Park Memorial Institute (RPMI) 1640 media and keratinocyte-SFM media (Gibco), respectively. Cells were passaged using 0.25% EDTA containing trypsin (Mediatech), and a hemacytometer was used to count cell number.

Peptide synthesis reagents (N^{α}—Fmoc protected amino acids, HBTU and HOBt) were purchased from either Anaspec or AAPPTec. The side-chain protecting groups used the following amino acids: Arg(N^{ω}-Pbf), Asn(N^{γ}-Trt), Glu(O-tBu), His(N^{im}-Trt), Ser(tBu), Lys(N^{ε}-Alloc), Lys(N^{ε}-Ivdde). Fmoc-AEA, Fmoc-AEEA, Fmoc-AE3 Å, Fmoc-AE6 Å Fmoc-AE10 Å and 6-aminohexanoic acid were purchased from AAPPTec. These reagents are analytical grade with >99% purity, and were used without further purification. Rink amide MBHA resin with initial loading of 0.2 mmol/g was acquired from Protein Technologies, Inc. Cy5.5-NHS ester was acquired from Lumiprobe. Analytical grade solvents for peptide synthesis and HPLC were purchased from Fisher Scientific, and were used without further purification unless otherwise stated.

### Example 4

### Synthesis and Labeling of Peptide Monomers and Peptide Heterodimers

Peptide monomers and heterodimers were synthesized and labeled with Cy5.5 using standard Fmoc solid-phase chemical synthesis with rink amide MBHA resin in a PS3 automatic synthesizer (Protein Technologies Inc). Fmoc protected L-amino acids were applied with standard HBTU/HOBt activation. The C-terminus lysine was incorporated as Fmoc-Lys(Alloc)-OH for fluorophore labeling. Upon completion of peptide assembly, the resin was transferred to a reaction vessel for manual labeling with dye. The Alloc side chain protecting group was removed with a palladium catalyst, as described previously. The resin was washed with dimethylformamide (DMF) and dichloromethane (DCM) for 1 min 3×. The protected resin-bound peptide was reacted with Cy5.5-NHS ester and (N,N-Diisopropylethylamine) DIPEA for 12 hours. The completion of the reaction was monitored with a qualitative ninhydrin test. A cleavage cocktail reagent TFA: TIS: H2O (95:2.5:2.5 v/v/v) was mixed with the resin, and stirred for 2 hours in dark conditions at 25° C. The crude peptides were isolated from the resin by filtration and evaporated with N2 gas followed by precipitation with chilled diethyl ether and stored at −20° C. for 12 hours. The precipitated peptides were centrifuged and washed 2× with ether, dried, dissolved in water, and lyophilized to produce a dark-green powder. The crude peptides were purified by prep-HPLC with a C18 column (Waters Inc.) using a water (0.1% TFA)-acetonitrile (0.1% TFA) gradient. Peptide purity was tested using an analytical C18-column. Further characterization was confirmed by either ESI (Waters Inc.) or Q-TOF (Agilent Technologies) mass spectrometry.

The absorbance spectra of the Cy5.5-labeled peptides were characterized with a spectrophotometer (NanoDrop 2000, Thermo Scientific). Fluorescence excitation and emission from a 1 μM peptide solution diluted in PBS was collected with a fiber coupled spectrophotometer (Ocean Optics) using a diode-pumped solid-state laser (Technica Laser Inc.) with λₑₓ=671 nm. The spectra were plotted with Origin 8.5 software (OriginLab Corp).

### Example 5

### Heterodimeric Peptide Configuration for Binding to EGFR and HER2

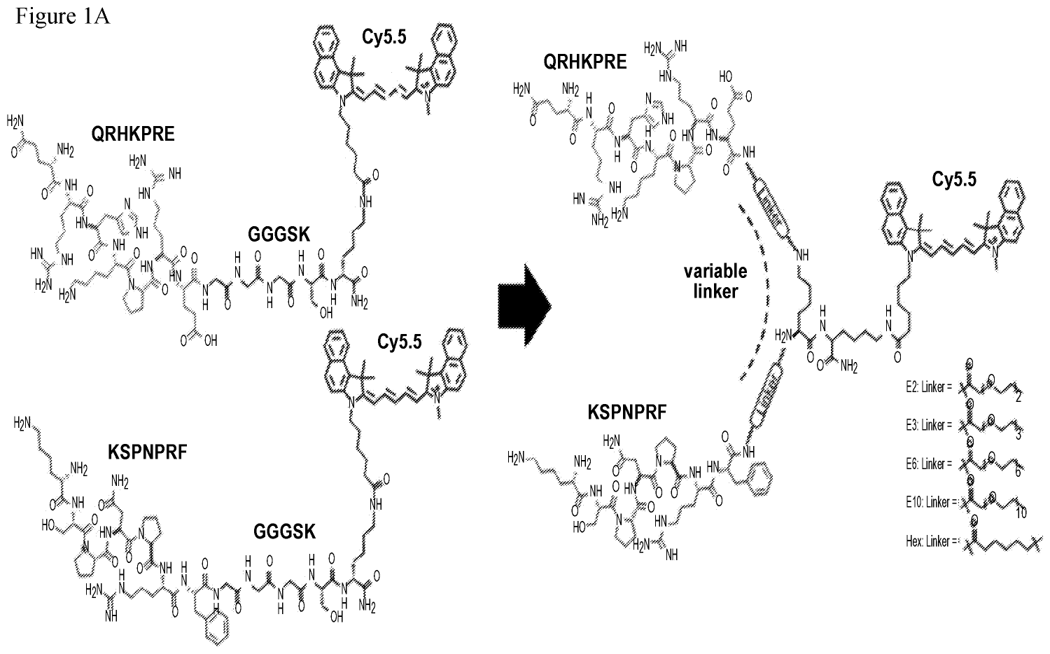

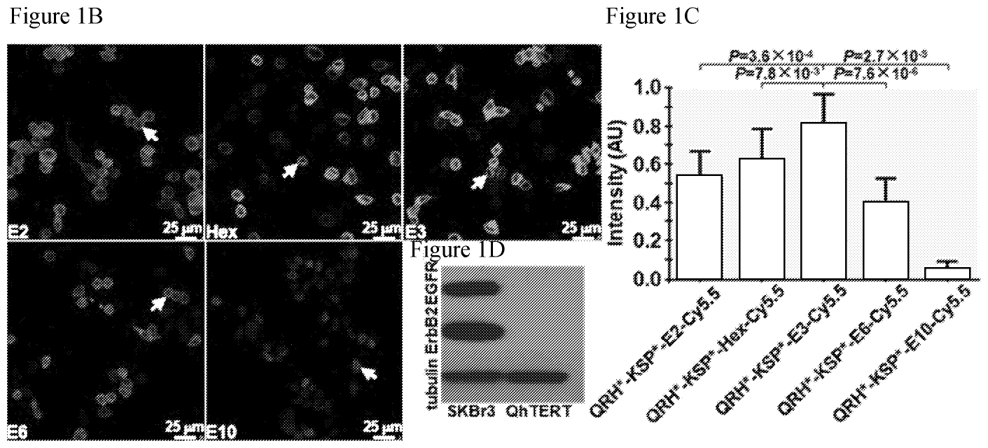

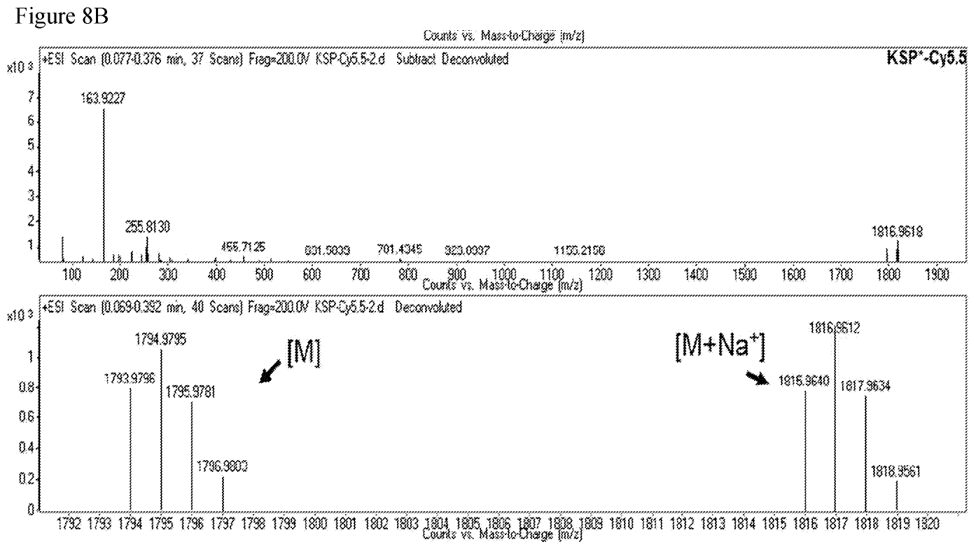

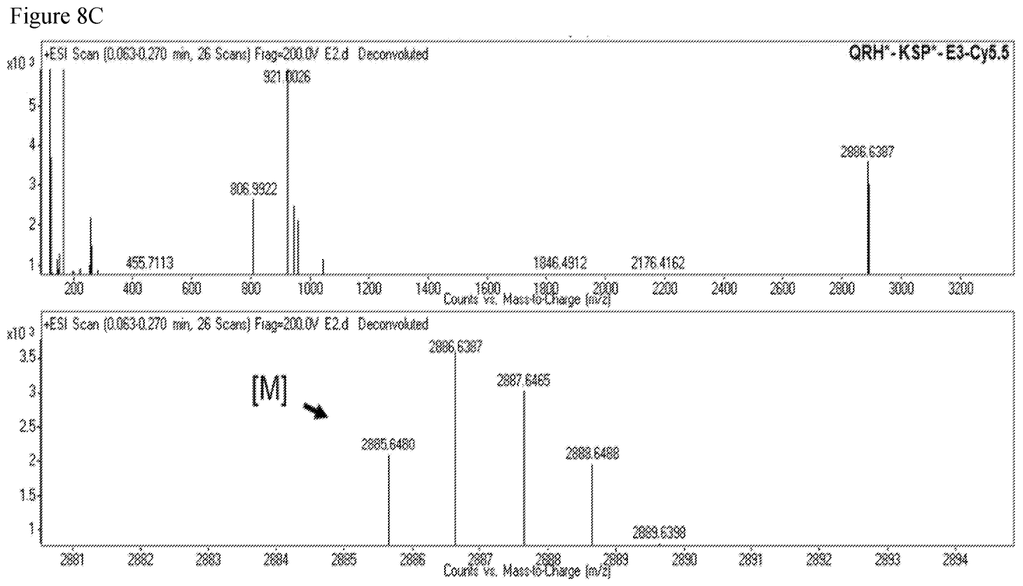

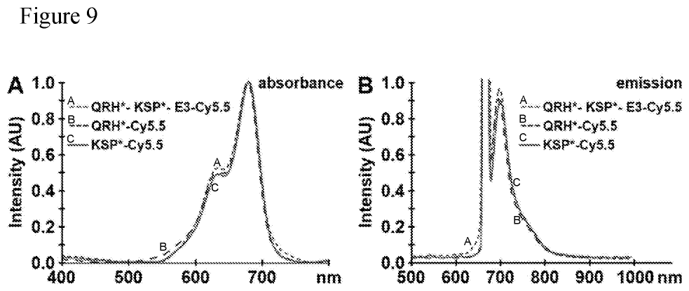

The seven-amino acid monomer peptides with sequence QRHKPRE (SEQ ID NO: 1) and KSPNPRF (SEQ ID NO: 2) were synthesized for specific binding to EGFR and ErbB2, respectively [Zhou et al., Clin. Transl. Gastroenterol., 6: e101, 2015); Joshi et al., Bioconjug. Chem., 27: 481-94, 2015]. A Cy5.5 fluorophore was attached to the C-termini via a GGGSK (SEQ ID NO: 3) linker on either peptide, hereafter QRH*-Cy5.5 and KSP*-Cy5.5, respectively, FIG. 1A. Cy5.5 was chosen for its photostability and high quantum yield in the near-infrared (NIR) spectrum [Luo et al. 32: 7127-38, 2011]. The linker separates the peptide and the fluorophore to prevent steric hindrance. For both peptides, >95% purity was achieved by analytical HPLC, and an experimental mass-to-charge (m/z) ratio of 1794.98 and 1900.04, respectively, was measured by mass spectrometry that agrees with the expected values, FIG. 8A,B. The fluorescence spectra for QRH*-Cy5.5 and KSP*-Cy5.5 shows peak absorbance and emission at λa=680 and λₑₘ=708 nm, respectively, FIG. 9A,B.

The two monomers were combined in a heterodimer configuration using a variable linker designed with either a PEG spacer that ranged in size from 17-74 atoms or a hydrophobic 6-aminohexonic acid linker in the α-, ξ-amino groups of the second lysine. The linker length was varied to determine the optimal spacing between monomers to maximize binding to the intended targets. Using confocal microscopy, binding for each Cy5.5-labeled heterodimer configuration to human breast cancer cells SKBr3 that overexpress both EGFR and ErbB2 was evaluated, FIG. 1B.

The mean (±SD) signal was quantified, and the heterodimer QRH*-KSP*-E3-Cy5.5 with the E3 PEG spacer showed the largest signal, FIG. 1C. Hydrophilicity was good as well. Western blot analysis was used to show relative EGFR and ErbB2 expression levels, FIG. 1D. Western blot was performed by lysing cells in Pierce® RIPA lysis buffer containing Halt™ protease inhibitor cocktail (#87786, Thermo Scientific) for 30 min on ice. The lysates were centrifuged at 10,000 rpm for 10 min at 4° C. A BSA protein assay kit (#23227, Thermo Scientific) was used to quantify protein concentration in the supernatant. Aliquots of protein (10 μL) were used for electrophoresis on NuPAGE™ 4-12% Bis-Tris gels (#17031471, Invitrogen) followed by electrophoretic transfer onto membranes (#ISEQ00010, Merck Millipore Ltd). The membranes were incubated with primary rabbit anti-EGFR monoclonal antibody (1:1000, #2232S) and rabbit anti-ErbB2 monoclonal antibody (1:1000, #2165S) from Cell Signaling Inc. Mouse anti-β-tubulin monoclonal antibody (1:500, #32-2600, Invitrogen) was used for loading control. For visualization, horseradish peroxidase (HRP)-conjugated secondary antibodies consisting of goat anti-rabbit IgG (H+L) HRP (1:5000, #65-6120) and goat anti-mouse IgG (H+L) HRP (1:5000, #62-6520) from Thermo Scientific were used, and followed by the ECL kit (#RPN2106, GE Healthcare) per manufacturer instructions.

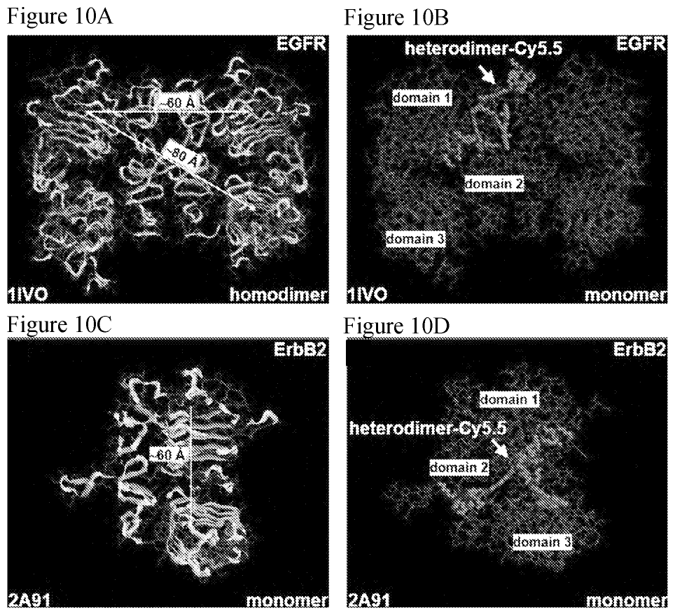

A structural model for EGFR (1IVO) and ErbB2 (2 Å91) was used to evaluate heterodimer binding, FIG. 10A-D. QRH*-KSP*-E3-Cy5.5 was found to bind domain 2 of EGFR with Eₜ=−656.47 and domain 3 of ErbB2 with Eₜ=−632.83. This configuration was synthesized for all further experiments, and obtained >95% purity by HPLC, and measured an experimental m/z ratio of 2974.69 by mass spectrometry that agrees with the expected value, FIG. 8C. The absorbance and emission spectra revealed no change in peak values in the heterodimer configuration, FIG. 9A,B.

The biochemical properties of peptide monomers and heterodimers are set out in Table 1 below.

TABLE 1
Biochemical properties of peptide monomers and heterodimers

| | Linker | Expt | Yield | Purity |
|---|---|---|---|---|
| Compound | (atoms) | Mass^{a} | (%) | (%)^{b} |
| QRH*-KSP*-E2-Cy5.5 | 17 | 2886.64 | 55.4 | >95% |
| QRH*-KSP*-Hex-Cy5.5 | 19 | 2822.66 | 38.3 | >95% |
| QRH*-KSP*-E3-Cy5.5 | 23 | 2974.69 | 40.2 | >95% |
| QRH*-KSP*-E6-Cy5.5 | 49 | 3266.88 | 30.1 | >95% |
| QRH*-KSP*-E10-Cy5.5 | 74 | 3619.09 | 35.2 | >95% |
| QRH*-GGGSK-Cy5.5 | — | 1794.98 | 61.8 | >95% |
| KSP*-GGGSK-Cy5.5 | — | 1900.04 | 50.9 | >95% |

^{a}Exact mass was calculated from most abundant isotope of element, and was characterized by Q-TOF mass spectrometry.
^{b}Analytical HPLC with C18-column using water (0.1% TFA) as gradient at 280 nm.

### Example 6

### Heterodimer Stability and Binding

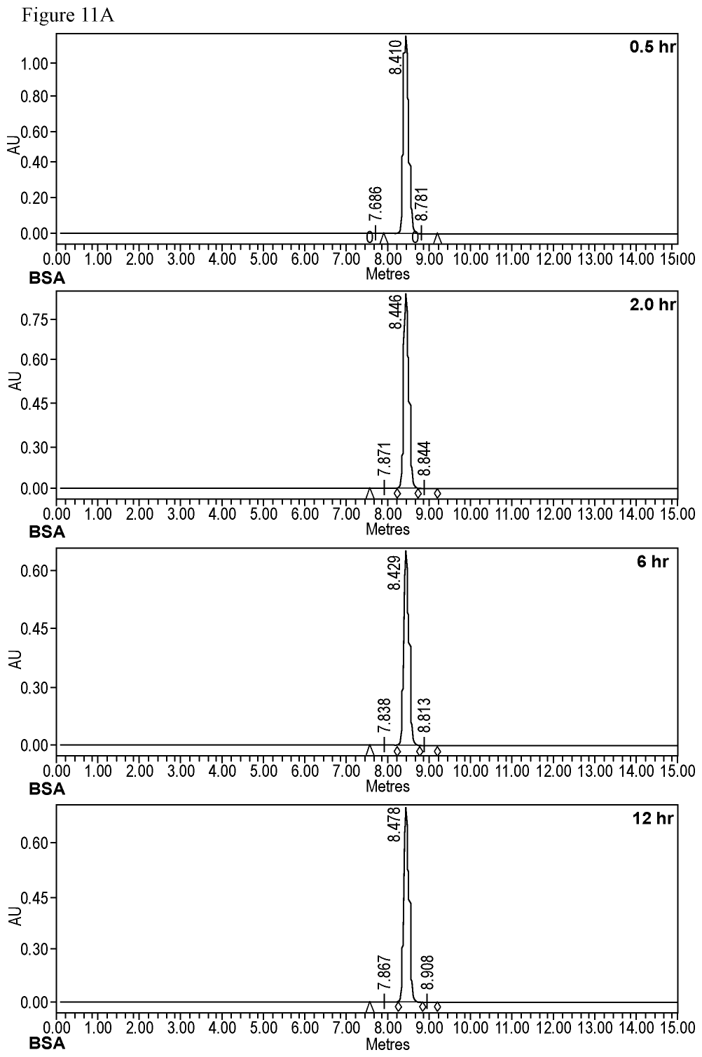

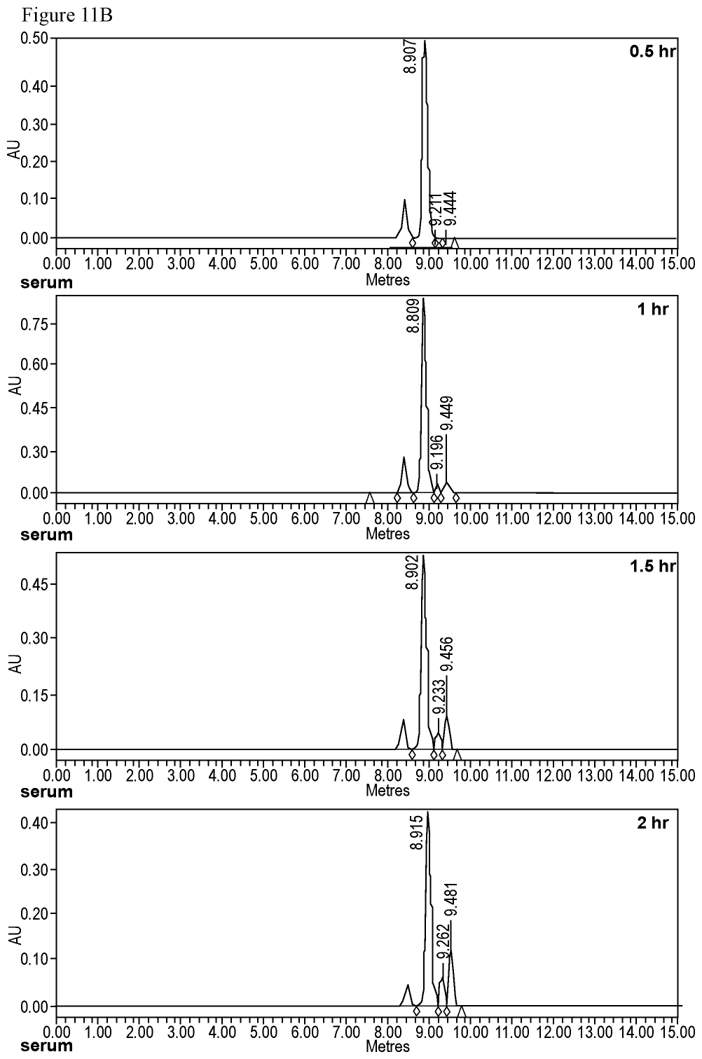

Heterodimer stability was evaluated in 0.01 mol/L PBS containing 0.1% bovine serum albumin (BSA, pH 7.4) at 37° C. Degradation was monitored by HPLC at about 0.5, 2, 6 and 12 hours. Enzymatic stability was also evaluated by incubating 30 μM of the heterodimer in mouse serum at 37° C. for about 0.5, 1.0, 1.5 and 2 hours. The samples were centrifuged at 14,000 rpm, and 20 μL aliquots of the supernatant were analyzed by HPLC at said incubation time points. No noticeable degradation was observed in either study, FIG. 11A,B.

The apparent dissociation constant (K_{d}) was measured to assess binding affinity of the heterodimer to OE33 cells. The heterodimer was serially diluted in PBS at concentrations of about 0, 10, 25, 50, 100, and 200 nM with about 105 OE33 cells at 4° C. for one hour and washed with cold PBS. The mean fluorescence intensities were measured with flow cytometry (BD LSRII, BD Biosciences). The equilibrium dissociation constant K_{d} 1/Kₐ was calculated with a least-squares fit of the data to the nonlinear equation I=(I₀+IₘₐₓKₐ[X])/(I₀+Kₐ[X]). I₀ and Iₘₐₓ are the initial and maximum fluorescence intensities, corresponding to no peptide and at saturation, respectively, and [X] represents the concentration of bound peptide. Prism 5.0 software (Graphpad Inc.) was used to calculate K_{d} and Kₐ.

The apparent association time constant (Kₐ) was measured to assess binding kinetics to OE33 cells. OE33 cells were grown to about 80% confluence in 10 cm dishes, and cells were then detached with PBS-based cell dissociation buffer (Invitrogen). About 10⁵ cells were incubated with 1 μM of the heterodimer at 4° C. for different time periods ranging from β-60 min. The cells were centrifuged, washed with cold PBS, and fixed with 4% PFA. Flow cytometry was performed, and the median fluorescence intensity (y) was ratioed with that of OE33 cells without addition of peptide at time points (t) using Flowjo software. The rate constant K was calculated by fitting the data to the first-order kinetics model, y(t)=Iₘₐₓ[1−exp^{(-kt)}], where Iₘₐₓ=maximum value, using Prism 5.0 software.

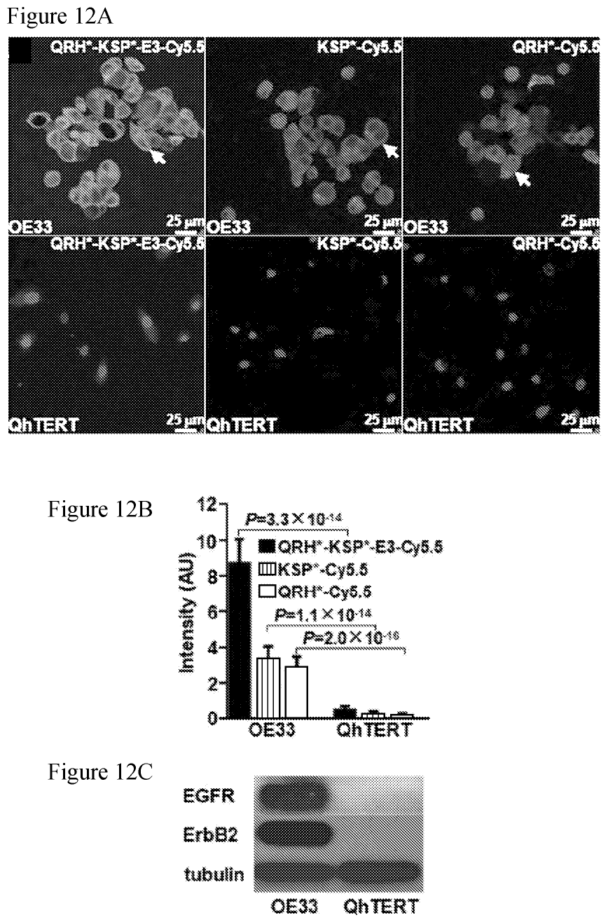

Using confocal microscopy, the peptide heterodimer (red) showed strong binding to the cell surface (arrow) of OE33 human esophageal adenocarcinoma cells, FIG. 12A. By comparison, less fluorescence intensity was seen with either monomer. Minimal binding was observed for any peptide to QhTERT human non-dysplastic Barrett's esophagus cells (control). Quantified results show the mean fluorescence intensity for the heterodimer to be significantly greater than that for either individual monomer, FIG. 12B. Western blot analysis showed EGFR and ErbB2 expression levels in OE33 and QhTERT cells, FIG. 12C.

Triethyleneglycol was identified as a linker that optimizes heterodimer binding to OE33 human EAC cells. The Cy5.5-labeled heterodimeric peptide, QRH*-KSP*-E3-Cy5.5, demonstrated 3-fold greater fluorescence intensity and 2-fold improved binding affinity compared with either monomer alone.

### Example 7

Specific Peptide Binding Using Small (or Short) Interfering RNA (siRNA) Knockdown

siRNA knockdown experiments were performed to further validate specific heterodimer binding to either EGFR or ErbB2 in vitro. Separate siRNA knockdown of either EGFR or ErbB2 expression in SkBr3 cells was carried out to validate specific peptide binding. ON-TARGET plus human EGFR siRNA (#L-003114-00-0005), ONTARGET plus human ErbB2 siRNA (#L-003126-00-0005) and ON-TARGET plus non-targeting pool (#D-001810-10-05) were used.

siRNA at 5 μM concentration in 5 μL was transfected into SKBr3 cells using DharmaFECT transfection reagents (Thermo Scientific). Briefly, cells were seeded in 6-well culture plates at 30% confluence in McCoy's 5 Å media supplemented with 10% FBS without antibiotics. The cells were transfected with siRNA at a final concentration of 5 M/L using oligofectamine (Thermo Scientific). Knockdown of either EGFR or ErbB2 was confirmed by Western blot.

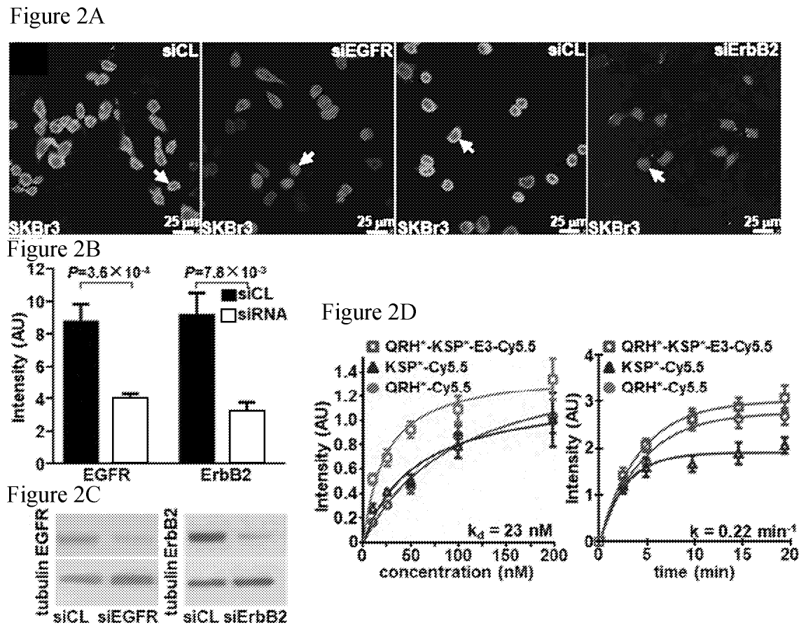

Using confocal microscopy, strong binding by QRH*-KSP*-E3-Cy5.5 (red) was observed to the surface (arrow) of SKBr3 cells transfected with siCL non-targeting siRNA (control), FIG. 2A. Less signal is seen with knockdown cells transfected with siEGFR targeting siRNA. Similar results were found for ErbB2. Quantified values show a significant reduction in mean fluorescence intensity for the heterodimer with either EGFR or ErbB2 in knockdown cells, FIG. 2B. Western blot analysis for control and knockdown SKBr3 cells is shown, FIG. 2C.

### Example 8

### Binding Properties

Binding parameters for the heterodimer were measured using flow cytometry with OE33 cells. An apparent dissociation constant of K_{d}=23 nM was found, FIG. 2D. This result showed that binding with the heterodimer was much improved compared with K_{d}=98 and 54 nM for monomers QRH*-Cy5.5 and KSP*-Cy5.5, respectively. Also measured was the apparent association time constant of Kₐ=0.22 min⁻¹ (4.5 min) for the heterodimer compared with Kₐ=0.21 min⁻¹ (4.7 min) and 0.35 min⁻¹ (2.8 min) for the monomers QRH*-Cy5.5 and KSP*-Cy5.5, respectively.

### Example 9

### Co-Localization and Heterodimer Internalization

Co-localization experiments were performed to validate specific heterodimer binding to EGFR and/or ErbB2 in SKBr3 cells. Cells were cultured to about 70% confluence on glass coverslips, washed with PBS, and incubated with 2% BSA in PBS for 30 min to block non-specific binding. 1 μM of Cy5.5-labeled peptides were added and incubated for 30 min or 1 hour at 4° C. The cells were then washed 3× with PBS, fixed with ice cold 4% paraformaldehyde (PFA) for 10 min, washed with PBS 1×, and then mounted on glass slides with ProLong Gold reagent containing DAPI (Invitrogen).

For antibody staining, cells were incubated with either anti-EGFR (1:500, #2232S) or anti-ErbB2 (1:500, #29D8) primary antibody from Cell Signaling Inc overnight at 4° C. after fixation, and then washed with PBS 3× and processed with secondary antibody staining. Either goat anti-rabbit IgG (H+L) labeled with Alexa-Fluor 488 (1:1000, #A-11008, Invitrogen) or goat anti-mouse IgG (H&L) labeled with Alexa-Fluor 568 (1:1000, #ab175473, Abcam) was added and incubated for 1 hour at room temperature (RT). The cells were washed with PBS 3×, and mounted onto glass coverslips. Confocal fluorescence images were collected using DAPI, AF488, AF568 and Cy5.5 filter sets. Fluorescence intensities from 3 independent images were quantified using custom Matlab (Mathworks) software.

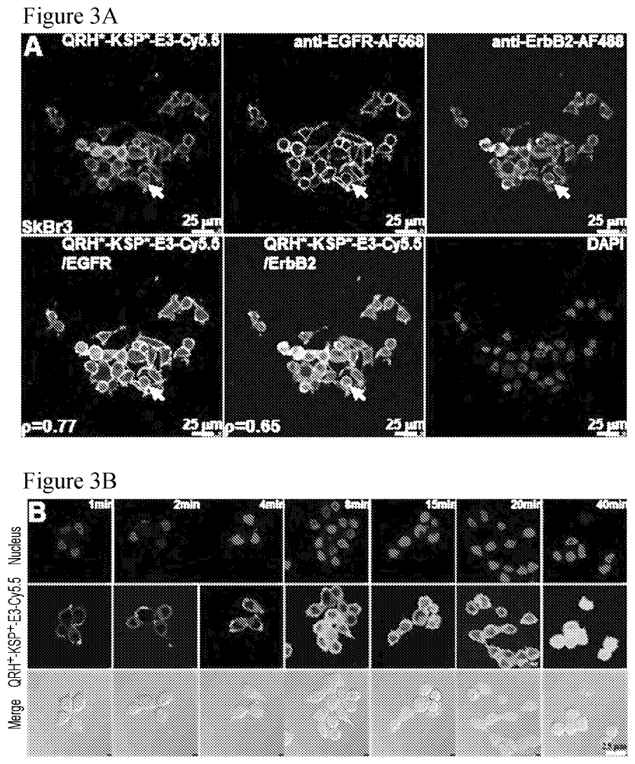

Using confocal microscopy, images were collected over time to evaluate heterodimer internalization. QRH*-KSP*-E3-Cy5.5 (red) was shown to bind strongly to the surface (arrow) of SKBr3 cells, FIG. 3A. Binding by anti-EGFR-AF568 (yellow) and anti-ErbB2-AF488 (green) to the surface (arrows) of the same cells is shown for comparison. On the merged images, a Pearson's correlation coefficient was measured for the heterodimer with the anti-EGFR antibody (ρ=0.77) and the anti-ErbB2 (ρ=0.65) antibody. The DAPI image shows the locations of the cell nuclei with no addition of either peptides or antibodies. The DAPI images highlight the location of cell nuclei prior to heterodimer incubation, FIG. 3B. Binding of QRH*-KSP*-E3-Cy5.5 (red) to the cell surface is seen at 1 and 24 min. The heterodimer starts to internalize at 4 min, and complete internalization is seen at 40 min.

### Example 10

### Effect of Heterodimer Binding on Cell Signaling

The effect of heterodimer binding on downstream signaling was evaluated in SKBr3 cells. SKBr3 cells were seeded in 6-well flat-bottom plates. For EGFR activation, serum-free media was used to culture the cells for 16 hours. EGF (#E9644, Sigma) was reconstituted to 1 mg/mL using 10 mM acetic acid, diluted with 0.1% BSA, and added to SKBr3 cells at a concentration of 100 ng/mL. Lapatinib (CDS022971, Sigma) was diluted to 100 nM in DMSO and PBS from 1 mg/mL stock solution, and was added to the cells incubated with heterodimer at concentrations of 1, 5, and 20 μM in separate wells. The cells were washed and harvested in RIPA buffer containing protease inhibitors (#11836170001, Roche), and evaluated by Western blot. Anti-EGFR antibody (#2232S), anti-ErbB2 antibody (#2165), anti-phospho-EGFR sampler kit (#9922s), anti-phospho-HER2/ErbB2 (Tyr1248) antibody (#2247), anti-AKT (#4691P), anti-ERK1/2 (#4695P), anti-phospho-AKT (pS473; #4060P), anti-phospho-ERK1/2 (#4370P) were obtained from Cell Signaling Inc. and were used per manufacturer's instructions.

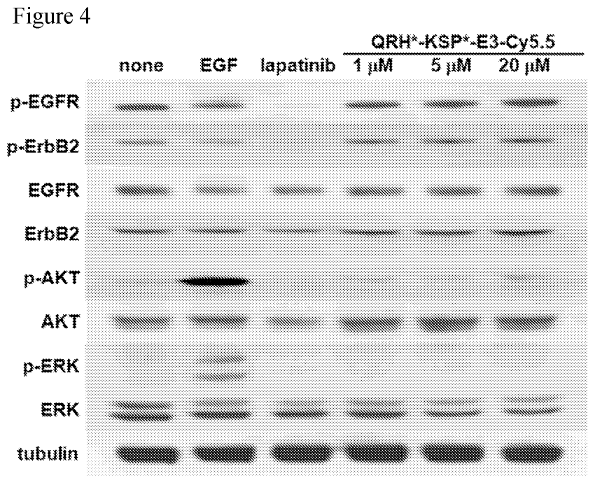

Western blot showed no change in phosphorylation of either EGFR2 (p-EGFR) or ErbB2 (p-ErbB2) with addition of 1, 5, and 20 μM of QRH*-KSP*-E3-Cy5.5, FIG. 4. Similarly, no downstream change was observed in AKT (p-AKT) and ERK (p-ERK). By comparison, strong phosphorylation activity of AKT (p-AKT) and ERK (p-ERK) was observed with addition of EGF as a positive control. Also, the addition of lapatinib, a dual tyrosine kinase inhibitor which interrupts the HER2/neu and epidermal growth factor receptor (EGFR) pathways, showed reduced expression of p-ErbB2, p-AKT, and p-ERK.

### Example 11

### Pharmacokinetics

Optical imaging was performed in nude mice bearing xenograft tumors in vivo to characterize pharmacokinetics and assess toxicity. All experimental procedures were performed in accordance with relevant guidelines and regulations of the University of Michigan, and all animal studies were conducted with approval by the University Committee on the Use and Care of Animals (UCUCA). Animals were housed per guidelines of the Unit for Laboratory Animal Medicine (ULAM).

Female athymic nude mice (002019 Foxn1, Jackson Laboratories) were obtained at 4-5 weeks of age, and were housed with four animals per cage and fed with sterilized pellet chow and water. Mice were anesthetized in an isoflurane chamber, and OE33 xenograft tumors were generated by subcutaneous injection of about 5×107 tumor cells suspended in 10 L of sterile PBS in the hind limb flank. Mice were studied at about 3-4 weeks post-inoculation when the tumor volume reached about 0.5-0.8 cm in size.

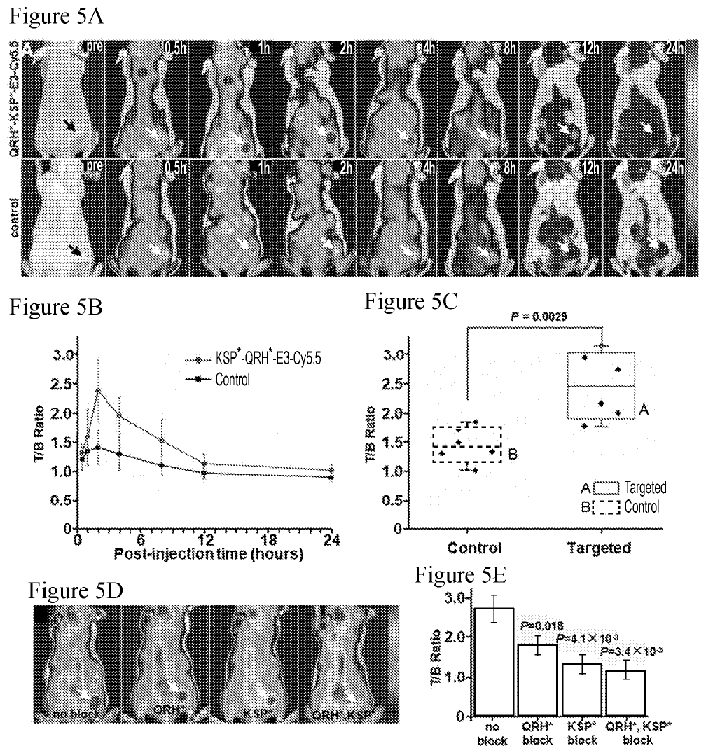

In vivo fluorescence images were collected using the Xenogen IVIS™ 200 small animal imaging system. Fluorescence intensities were measured and normalized. Mice were anesthetized using isoflurane, and 300 μM of either the heterodimer QRH*-KSP*-E3-Cy5.5 or control (GGGAGGG)₂KK(SEQ ID NO: 7)-Cy5.5, diluted in 150 μL of PBS, were administered via the tail vein. The heterodimer or control was injected via tail vein to evaluate in vivo uptake by OE33 xenograft tumors implanted in nude mice. Images were collected prior to peptide injection to assess baseline auto-fluorescence values and then at various time points post-injection up to 24 hours, FIG. 5A. Fluorescence images were excited at λₑₓ=675 nm, and emission was collected at λₑₘ=740 nm with 5 sec exposure (f/stop=8). Peak tumor uptake of the heterodimer was observed in mouse xenograft tumors at 2 hours post-injection with systemic clearance by about 24 hours, FIG. 5B.

### Example 12

### Assessing Heterodimer Specificity Via Competition

In vivo heterodimer specificity was assessed by competition with 3000 μM of unlabeled peptide monomer or a mixture of two monomers diluted in 50 μL of PBS injected 20 min beforehand in tumor-bearing mice (n=4 per group). Blocking studies were carried out by injecting unlabeled QRH*, KSP*, and both monomers together to validate in vivo specificity. Images were collected at two hours after heterodimer injection when peak uptake was expected. The ratio of fluorescence intensity in tumor was calculated using regions of interest (ROI), and the results were presented as mean±standard deviation (SD).

A significant reduction in fluorescence intensity from the tumor at about two hours post-injection was observed with each condition, FIG. 5C. Quantified results are shown, FIG. 5D.

### Example 13

### Biodistribution of Heterodimer Uptake

The biodistribution of heterodimer uptake, i.e., peptide biodistribution, was evaluated in nude mice bearing OE33 xenograft tumors following systemic injection. Each mouse (n=4) was injected with either the heterodimer QRH*-KSP*-E3-Cy5.5 or control peptide (GGGAGGG)₂KK(SEQ ID NO: 7)-Cy5.5 (at a concentration of 300 μM diluted in 150 μL of PBS), and euthanized about two hours later. In the control peptide, the two lysines are connected at the C-terminus of each glycine, and the two lysines are connected via a peptide bond. Organs were harvested, separated, and imaged ex vivo using the Xenogen IVIS™ 200 small animal system. Fluorescence intensity from each organ was quantified using regions of interest, and the results were presented as mean±standard deviation (SD).

The fluorescence intensities from the human esophageal specimens were fit according to 4 histological classifications, including squamous (SQ), Barrett's esophagus (BE), high-grade dysplasia (HGD), and esophageal adenocarcinoma (EAC), and used a one-way ANOVA and used Tukey's multiple comparisons. Co-localization of peptide and antibody binding was evaluated using Pearson's correlation coefficient.

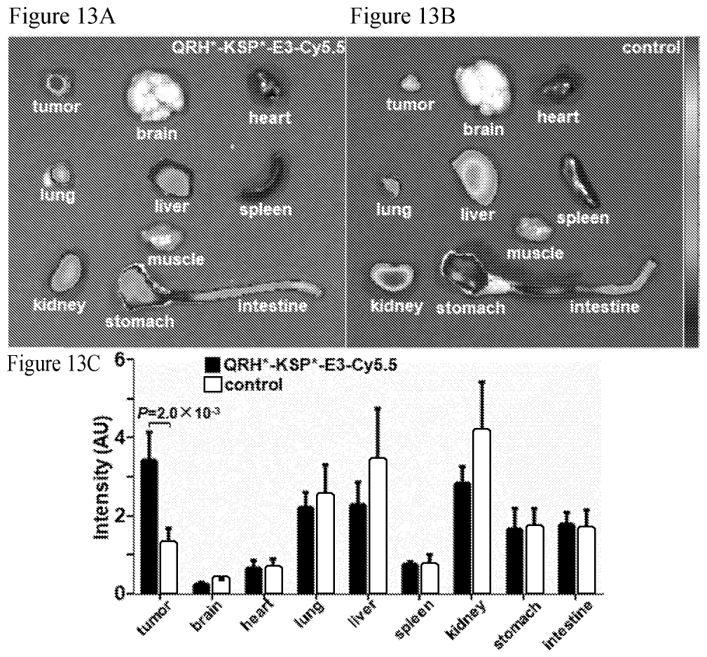

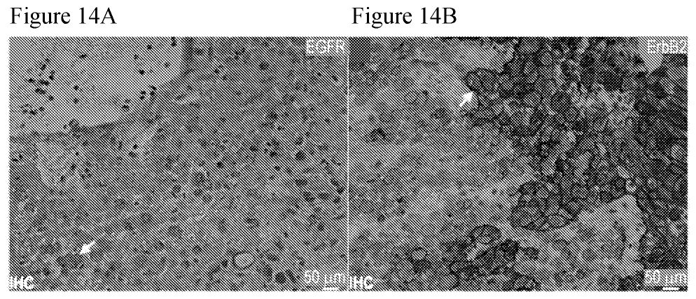

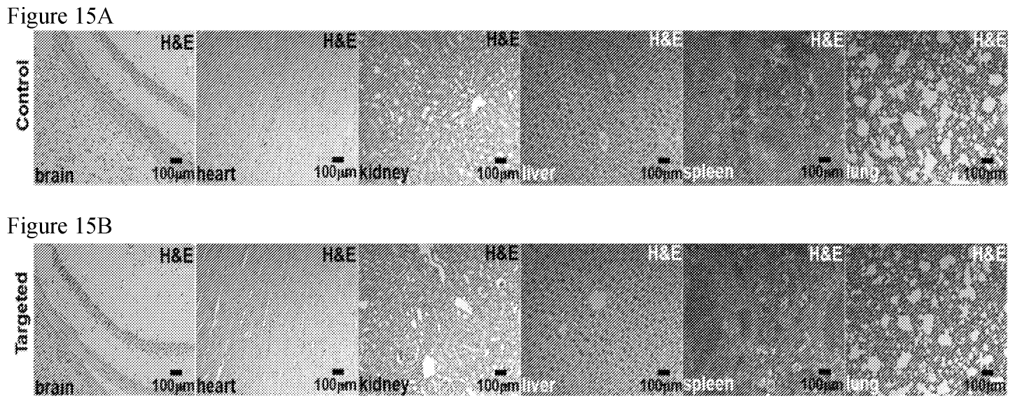

Heterodimer uptake was significantly higher in the tumors than in other organs, including about five-fold higher than in muscle, FIG. 13A. High uptake was also visualized in kidney to support a renal clearance mechanism. Significantly less intensity, i.e., less uptake, was observed in tumor using the control peptide, FIG. 13B. The quantified results show a significantly greater mean value in tumor for the heterodimer versus control, FIG. 13C. Expression of EGFR and ErbB2 was validated in these sections ex vivo with immunohistochemistry (IHC), FIG. 14A,B. No evidence of acute toxicity was found on histology (H&E), FIG. 14.

### Example 14

Validation with Human Esophageal Specimens

Using confocal microscopy, specific heterodimer binding to Barrett's neoplasia was validated on human specimens ex vivo. Formalin-fixed, paraffin-embedded (FFPE) specimens of human esophagus were obtained from the archived tissue bank in the Department of Pathology, University of Michigan. The tissues were cut into 10 m sections, deparaffinized, and rehydrated using standard conditions. Two-step acidic and protease-based antigen retrieval was performed. The sections were blocked with protein serum for 10 min at RT followed by rinsing with PBS, and then incubated with the heterodimer (0.5 μM) for ten min in dark conditions at RT. The sections were then washed for three min 3× with PBS and further incubated sequentially with 1:200 dilution of EGFR monoclonal antibody (H11, #MA5-13070, Invitrogen) and 1:500 goat anti-mouse IgG (H+L) AF-568-labeled secondary antibody (#A-11004, Thermo Fisher Scientific), 1:500 dilution of ErbB2 primary antibody (#29D8, rabbit monoclonal antibody #2165S, Cell Signaling Inc), and 1:1000 goat anti-rabbit IgG (H+L) AF-488-labeled secondary antibody (#A11008, Life Technologies Corp). Each antibody was incubated for 1 hour at RT. Sections were washed with TBST 3× and mounted with Prolong Gold reagent containing DAPI (Invitrogen) using #1 cover glass (1.5 m thickness). Confocal fluorescence microscopy was performed using DAPI, AF488, AF568 and Cy5.5 filter sets. Image quantification was performed by placing 3 boxes with dimensions of 20×20 μm² completely within cells found in the epithelium. Mean fluorescence intensities were measured using custom Matlab software (Mathworks). Regions of saturated intensities were avoided.

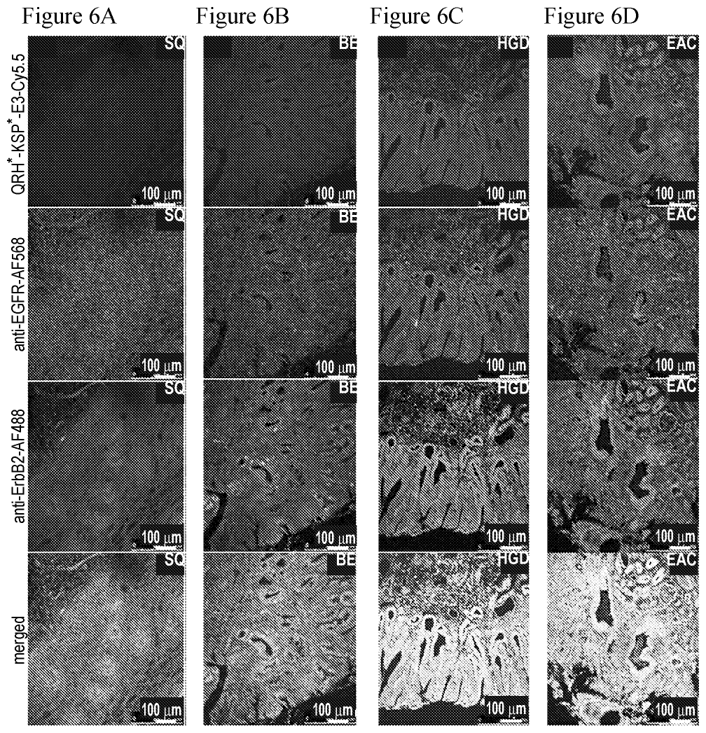

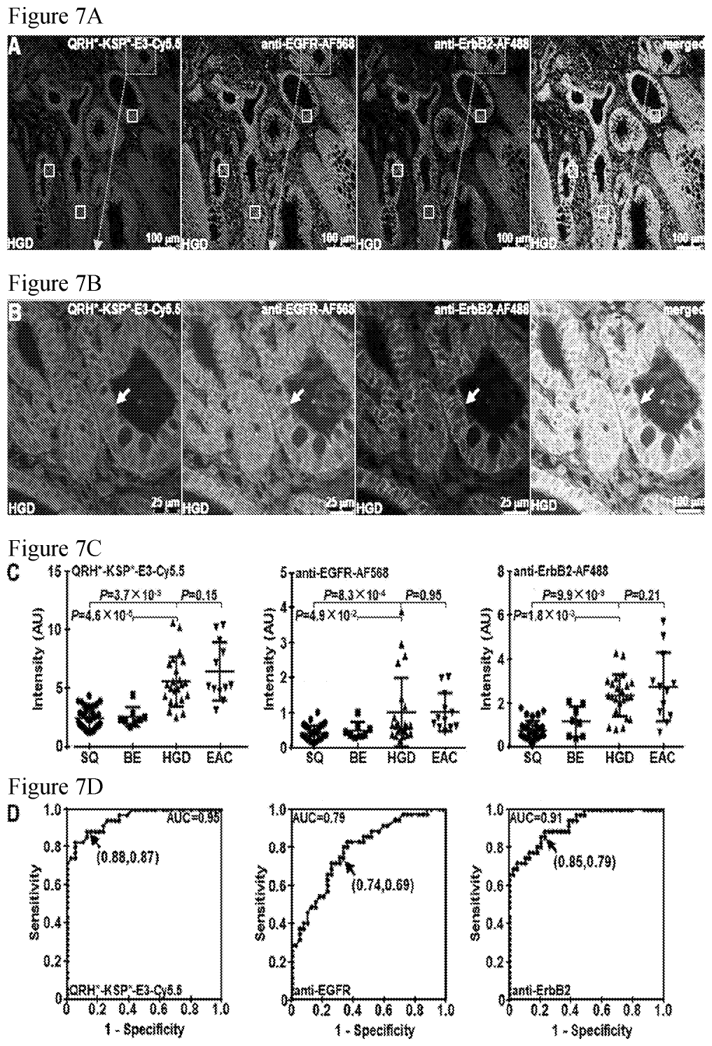

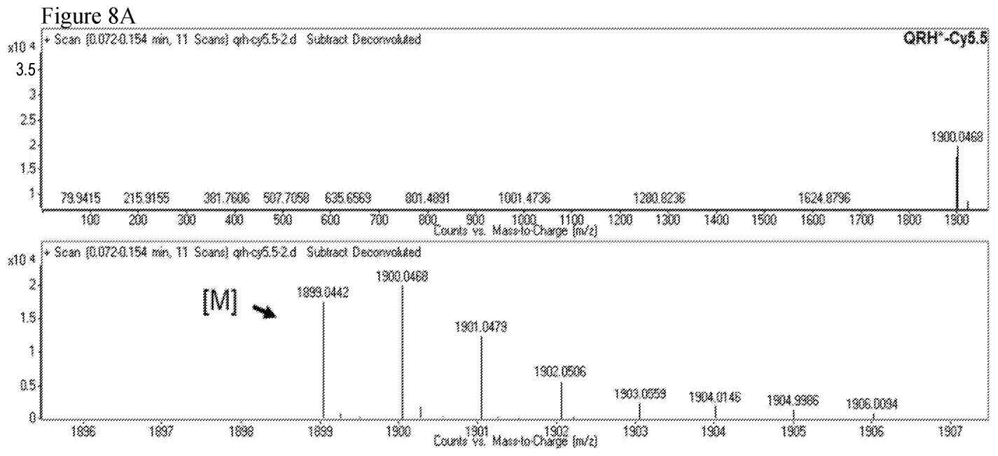

Minimal fluorescence signal, i.e., intensity, was observed with SQ and BE, and increased signal was observed with HGD and EAC, FIG. 6A-D. The same set of specimens was also stained with AF568-labeled anti-EGFR antibody (yellow), and AF488-labeled anti-ErbB2 antibody (green). Heterodimer and antibody binding co-localized on the merged images. Fluorescence intensities were measured from a set of three boxes with dimensions of 30×30 μm² placed within random crypts of heterodimer and antibody images, FIG. 7A. At higher/greater magnification, cell surface binding (arrows) was appreciated, FIG. 7B. Binding co-localization between the heterodimer QRH*-KSP*-E3-Cy5.5 (red) and the anti-EGFR-AF568 (yellow) and anti-ErbB2-AF488 (green) antibodies was measured, and Pearson's correlation coefficients of ρ=0.60 and 0.75, respectively, were found.

Mean (±SD) fluorescence intensity was measured for the heterodimer from n=31, 8, 23, and 12 human specimens of SQ, BE, HGD, and EAC, respectively, FIG. 7C. A significantly higher fluorescence intensity was measured for HGD than for BE and SQ. Similar findings were observed with the anti-EGFR and anti-ErbB2 antibodies. From receiver-operator characteristic (ROC) curves, 88% sensitivity and 87% specificity were found for the heterodimer to detect Barrett's neoplasia (HGD and EAC) at a T/B ratio of 5.9 when compared with pathology, FIG. 7D. The sensitivity and specificity for the heterodimer was higher than that for either antibody alone. Herein, a heterobivalent peptide ligand that binds EGFR and ErbB2 has been demonstrated for targeted detection of early Barrett's neoplasia and other esophageal diseases. The data also confirms that this heterodimeric peptide recognizes and binds two different cell surface targets that are validated EAC biomarkers. Thus, the peptide heterodimer provides a new means for improved targeting in the early detection and therapy of Barrett's neoplasia, esophageal adenocarcinoma (EAC), high grade dysplasia (HGD) of the esophagus, or other abnormality or cancer of the esophagus.

### Example 15

### Dual-Modal In Vivo Fluorescence and Photoacoustic Imaging Using Heterodimeric Peptide

The heterodimeric peptide (QRH*-KSP*) labeled with IRDye800 was used to perform dual-modal imaging of human esophageal xenograft tumors in vivo. Fluorescence and photoacoustic images provide complementary visualization of tumor dimensions in planar and sagittal views, respectively, demonstrating utility in targeted cancer diagnosis and staging. Multi-modal imaging uses the physical properties of one modality to confirm the results of another to provide rigorous in vivo validation. This may lead to more accurate assessment of tumor margins for image-guided surgery to maximize cancer excision and minimize resection of normal tissues. Fluorescence (FL) imaging offers high contrast, but has limited tissue penetration. Photoacoustic (PA) imaging combines light excitation with sound detection to increase imaging depth on the centimeter scale. Combining these two imaging modalities provides ability to improve diagnostic and therapeutic performance.

Here, IRDye800 was used to label the heterodimeric peptide for dual-modal fluorescence and photoacoustic imaging with a single contrast agent. This near-infrared (NIR) fluorophore absorbs and emits light in a spectral band least affected by hemoglobin absorption, tissue scattering, and autofluorescence and, has optimal tissue penetration depth (Smith et al., Nat. Nanotechnol. 4:710 (2009)).

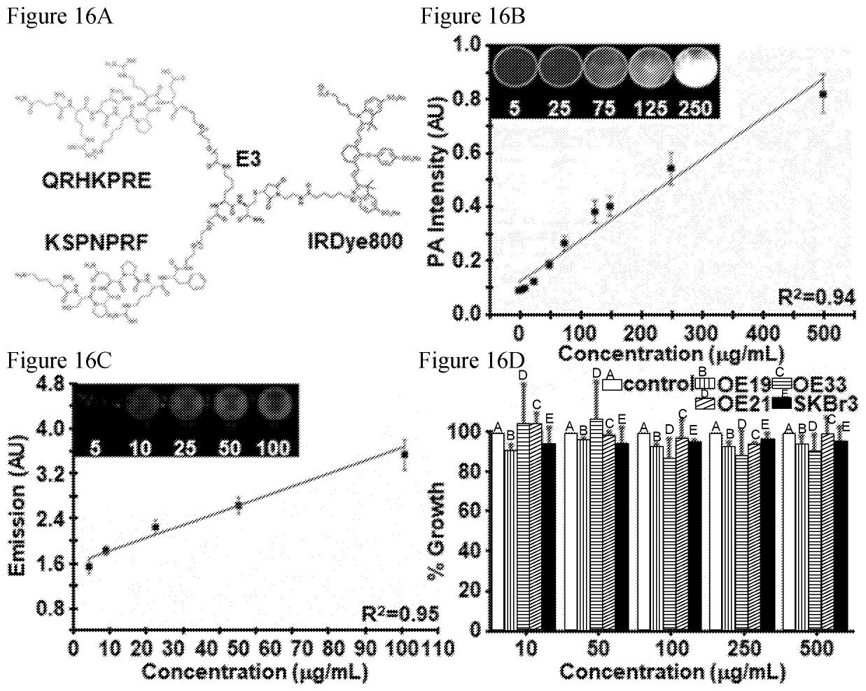

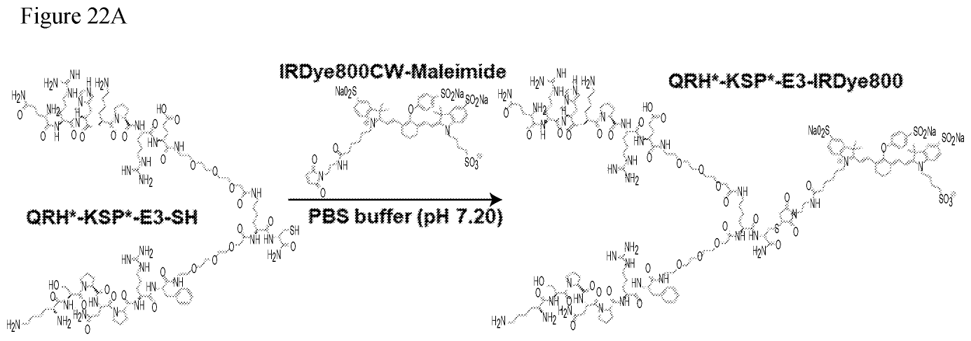

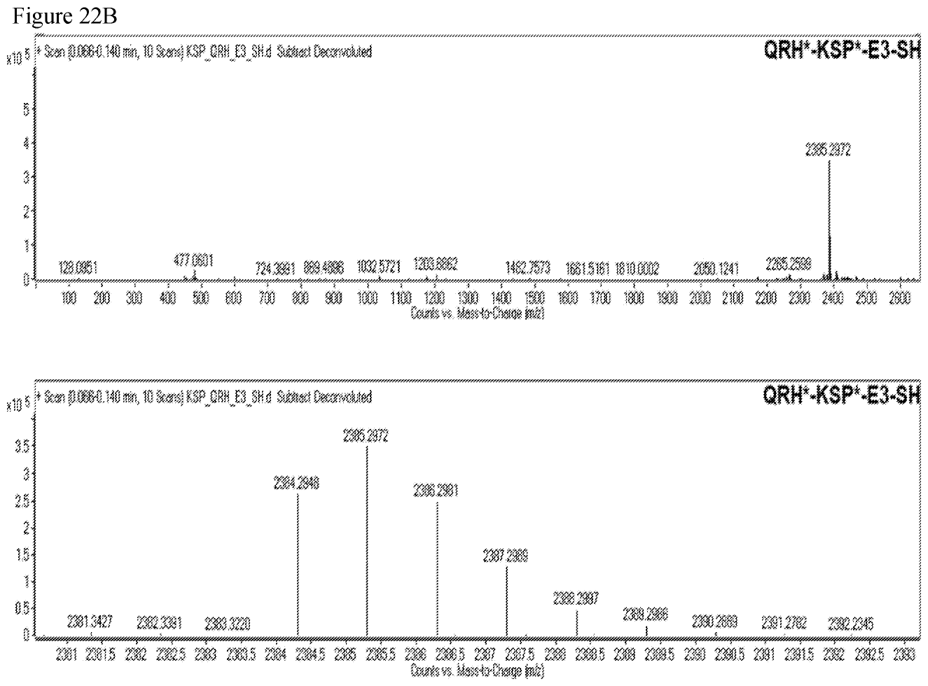

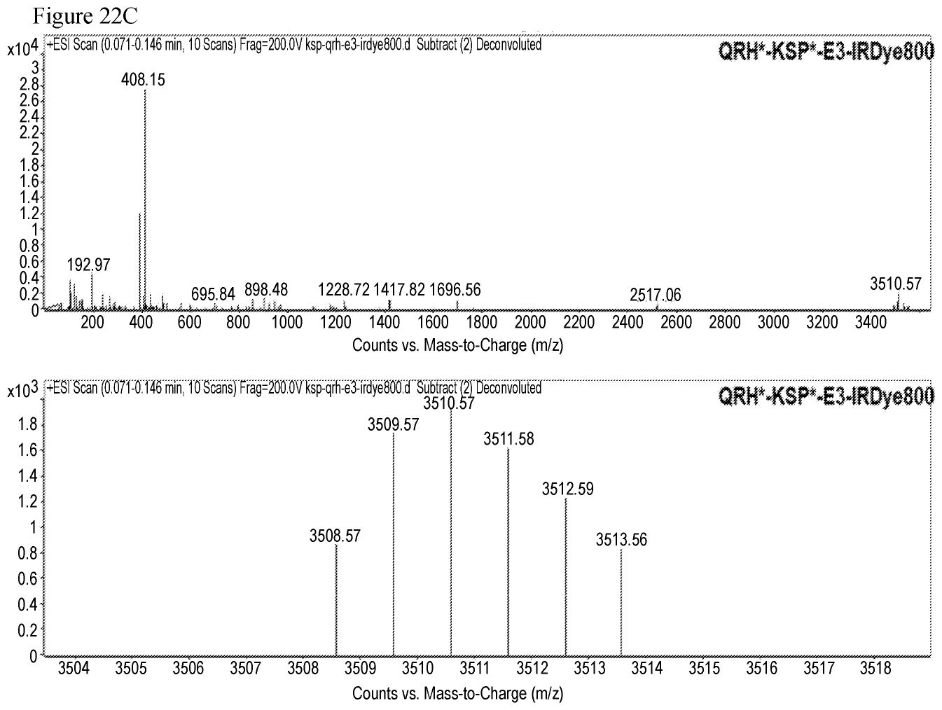

The heterodimer was assembled by arranging QRH* and KSP* with an E3 spacer, as discussed herein above, to avoid steric hindrance from the label and interactions between monomers, FIG. 16A. IRDye800 has been found to be safe in animals (Marshall et al., Mol. Imaging Biol. 12:583-594 (2010)) and has been used in human clinical studies (Lamberts et al., Clin. Cancer Res. 23:2730-41 (2017); Miller et al., 139:135-43 (2018); Rosenthal et al., (Clin. Cancer Res. 21:3658-66 (2015)); Heath et al., Otolaryngol. Head Neck Surg. 148:982-90 (2013); Heath et al., Ann. Surg. Oncol. 19:3879-87 (2012)). This fluorophore has two solubilizing sulfonated groups, and remains extracellular rather than cross the cell membrane, reducing non-specific binding interactions. QRH*-KSP*-E3-IRDye800 was purified with preparative HPLC for >95% purity on analytical reverse phase HPLC (RP-HPLC). A mass-to-charge (m/z) ratio of 3508.57 was measured using mass spectrometry. This result agrees with the expected value of 3508.06. FIG. 22A-C.

The peak values for optical absorbance and fluorescence emission of QRH*-KSP*-E3-IRDye800 and (GGGAGGG (SEQ ID NO: 28))2-E3-IRDye800 (control) peptide were found to be λab=780 and λem=795 nm, respectively, and maximum PA intensity was λab=770 nm. The relationship between PA intensity and FL emission with heterodimer concentration was found to be linear over a large range of concentrations, FIG. 16B,C, allowing for image intensities to be quantified and compared. Negligible cytotoxicity was observed with a panel of human cancer cells in vitro, FIG. 16D.

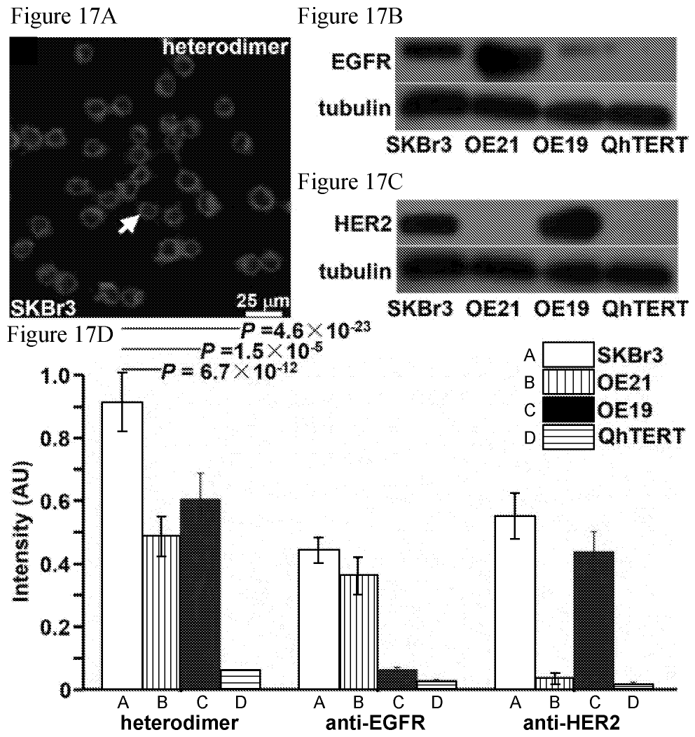

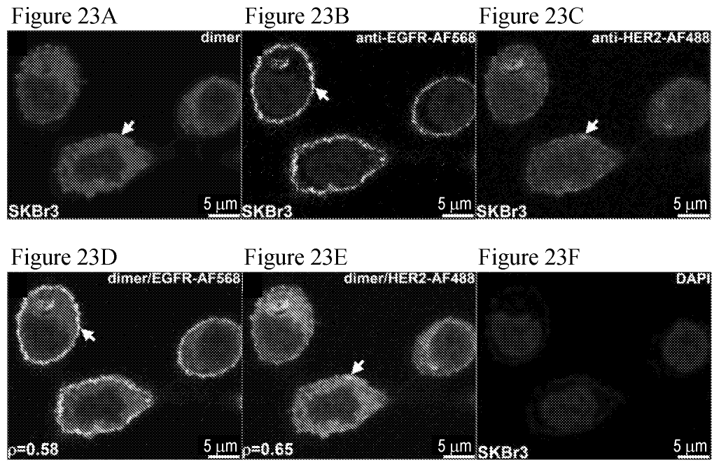

Confocal microscopy was performed to validate specific heterodimer binding. Strong fluorescence signal was observed for the heterodimer to the surface (arrow) of SKBr3 human breast cancer cells that express EGFR and HER2, FIG. 17A. Western blot shows EGFR and HER2 expression by a panel of human cancer cells, FIG. 17B,C. Quantified results show that the mean fluorescence intensity from the heterodimer with SKBr3 cells is significantly greater than that with OE21, OE19, and QhTERT cells that do not express both EGFR and HER2, FIG. 17D. AF568-labeled anti-EGFR and AF488-labeled anti-HER2 antibodies were used as positive controls to confirm EGFR and HER2 expression by the cell panel. Heterodimer and antibody binding to the surface (arrows) of SKBr3 cells co-localize, and a Pearson's coefficient of ρ=0.58 and 0.65 was measured for EGFR and HER2, respectively, on merged images, FIG. 23A-F.

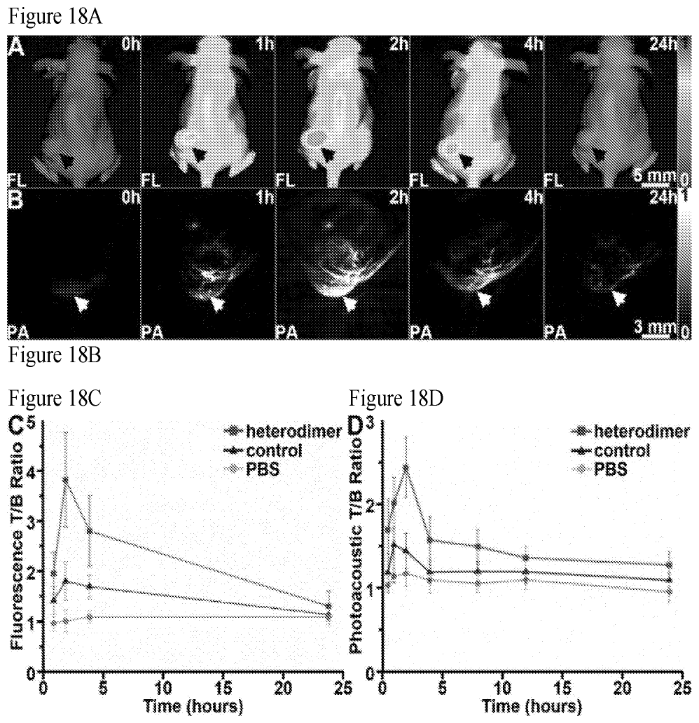

FL and PA images were collected following systemic administration of QRH*-KSP*-E3-IRDye800 and (GGGAGGG (SEQ ID NO: 28))2-E3-IRDye800 (control) in human OE33 xenograft tumors to characterize pharmacokinetics. Prior to injection (0 hour), minimal FL and weak PA signal was observed from tumor (arrow), FIG. 18A,B. FL and PA intensities from tumor peaked at 2 hours following heterodimer injection, FIG. 18C,D. The mean target-to-background (T/B) ratio was significantly greater for the heterodimer than either the control peptide or PBS in both FL and PA images. The mean intensity for either modality regressed to baseline at ˜24 hours post-injection.

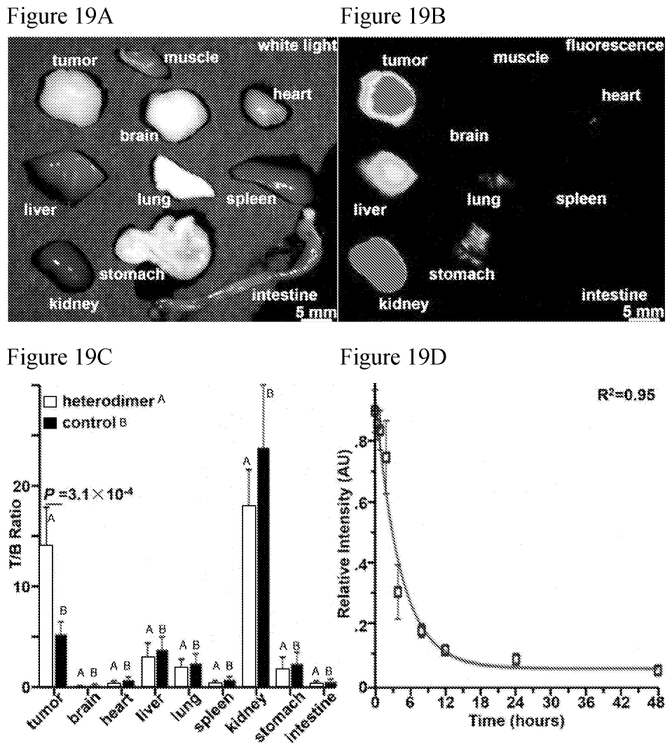

Tumor-bearing mice were sacrificed at 2 hours post-injection of heterodimer or control peptide. White light and NIR FL images were collected from major organs, FIG. 19A,B. Heterodimer uptake was found to be significantly higher in tumor versus control peptide, FIG. 19C. Low uptake was observed in all other organs except kidney, where the peptide was excreted. The relative concentration of heterodimer in the serum of tumor bearing mice was found to decrease from 90.1 to 1.33% over 48 hours with a half-life of ˜3 hours, FIG. 19D.

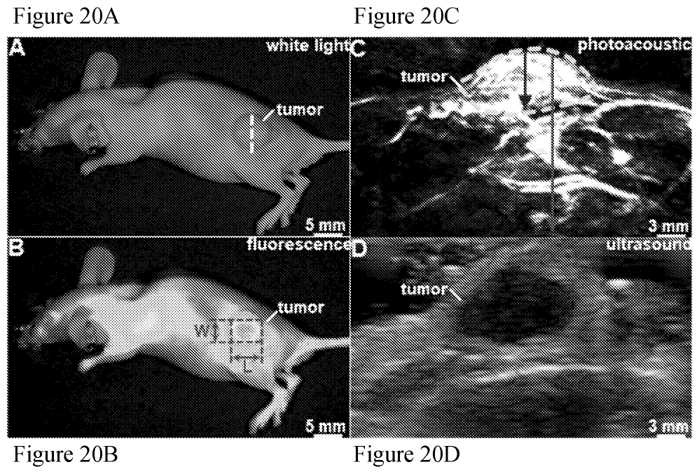

Dual-modal images were collected at 2 hours following heterodimer injection to assess imaging depth. A representative white light image shows the location of tumor, FIG. 20A. The corresponding FL image shows the tumor highlighted in pseudocolor, FIG. 20B. A PA image of the same tumor shows a depth of 4.8 mm (blue arrow) with a total imaging depth of 1.2 cm (red arrow) on sagittal view, FIG. 20C. An ultrasound image confirmed tumor structure, FIG. 20D.

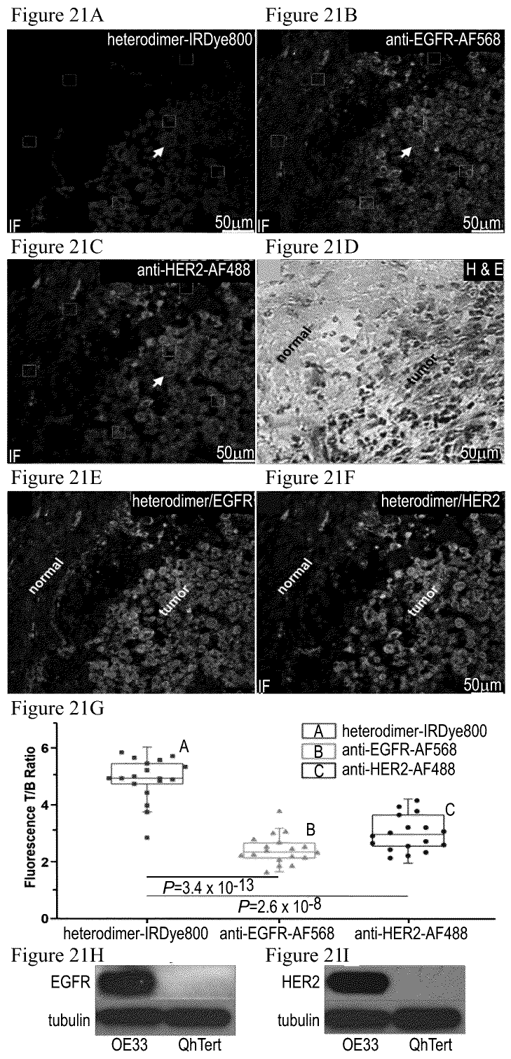

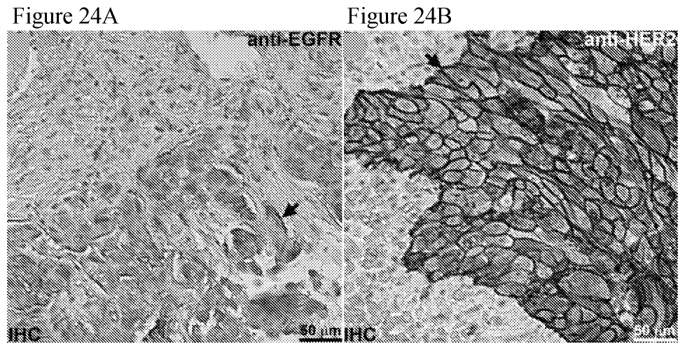

Immunofluorescence was performed to validate target expression in sections of OE33 human xenograft tumor ex vivo. Strong FL intensity was observed with the heterodimer (red), AF568-labeled anti-EGFR (yellow), and AF488-labeled anti-HER2 (green) to the surface (arrows) of tumor cells, FIG. 21A-C. Representative histology (H&E) showed the boundary between normal and tumor, FIG. 21D. Differences in EGFR and HER2 expression on either side of the margin can be appreciated on the merged images, FIG. 21E,F. Quantified results show a significantly higher T/B ratio for the heterodimer than for either anti-EGFR-AF568 or anti-HER2-AF488, FIG. 21G. Immunohistochemistry results confirm cell surface (arrow) expression of EGFR and HER2 in OE33 tumor sections, FIG. 24A-B. Western blot shows expression of EGFR and HER2 by OE33 and QhTERT cells, FIG. 21H-I.

Here, dual-modal imaging was used to characterize a heterodimeric peptide. Specific binding of QRH*-KSP*-E3-IRDye800 to human cancers cells that express EGFR and HER2 was validated. In vivo images collected in human xenograft tumors show peak uptake at ˜2 hours for both modalities. Clearance occurs by ˜24 hours, and the biodistribution supports high tumor uptake. Serum stability results were consistent with a half-life of ˜3 hours. Tissue imaging depth of 1.2 cm was observed with photoacoustic imaging. Sharp tumor margins were observed using immunofluorescence, and target expression was validated with immunohistochemistry. No cytotoxicity was found with cells.

Accurate diagnosis and staging of cancer is critical to determine appropriate therapy and obtain good outcomes. FL imaging provides high contrast in a planar view, and can be useful to delineate tumor margins. PA imaging has utility in assessing mucosal invasion by esophageal cancer and evaluating the extent of lymph-node involvement (Yang et al., Nat. Med. 18:1297 (2012)). The data provided herein above demonstrated use of QRH*-KSP*-E3-IRDye800, a NIR-labeled heterodimeric peptide specific for EGFR and HER2, for dual FL and PA imaging of human xenograft tumors in vivo. The combination of FL and PA imaging are complementary, and can be used together for diagnosis, staging, and therapy of esophageal cancer.

While the disclosure has been described in terms of specific embodiments, it is understood that variations and modifications will occur to those skilled in the art. Accordingly, only such limitations as appear in the claims should be placed on the disclosure.

All documents cited in this application are hereby incorporated by reference in their entirety, with particular attention to the disclosure for which they are referred.

The disclosure has described various amino acid and nucleic acid sequences, which are provided herein below in Table 2.

TABLE 2
SEQUENCE TABLE

| SEQ ID NO: | SEQUENCE |
|---|---|
| 1 | QRHKPRE |
| 2 | KSPNPRF |
| 3 | GGGSK |
| 4 | PPSNFKR |
| 5 | NRHKPREKTFTD |
| 6 | PEHKRRQ |
| 7 | GGGAGGGGGGAGGGKK |
| 8 | ccctcatagttagcgtaacg |
| 9 | HAHRSWS |
| 10 | YLTMPTP |
| 11 | TYPISFM |
| 12 | KLPGWSG |
| 13 | IQSPHFF |
| 14 | YSIPKSS |
| 15 | SHRNRPRNTQPS |
| 16 | TAVPLKRSSVTI |
| 17 | GHTANRQPWPND |
| 18 | LSLTRTRHRNTR |
| 19 | RHRDTQNHRPTN |
| 20 | ARHRPKLPYTHT |
| 21 | KRPRTRNKDERR |
| 22 | SPMPQLSTLLTR |
| 23 | NHVHRMHATPAY |
| 24 | RHPFPRF |
| 25 | RHPWPNR |
| 26 | RHPYPQR |
| 27 | RKPFPRH |
| 28 | GGGAGGG |
| 29 | GGGAGGGK |

1. A reagent comprising:
(a) a heterodimeric peptide comprising an epidermal growth factor receptor (EGFR)-specific peptide QRHKPRE (SEQ ID NO: 1) and an epidermal receptor growth factor 2 (ErbB2)-specific peptide KSPNPRF (SEQ ID NO: 2), or a multimeric form of the heterodimeric peptide, wherein the heterodimeric peptide specifically binds to EGFR and ErbB2; and
(b) at least one detectable label, at least one therapeutic moiety, or both, wherein the label, the therapeutic moiety, or both, are attached to the heterodimeric peptide or the multimeric form of the heterodimeric peptide.

2. The reagent of claim 1, wherein the EGFR-specific peptide and the ErbB2-specific peptide are joined by one or more linkers.

3. The reagent of claim 1, wherein the detectable label is attached to the peptide by a linker.

4. (canceled)

5. The reagent of claim 1, wherein the linker is attached at the C-terminus of the EGFR-specific peptide and at the C-terminus of the ErbB2-specific peptide.

6. The reagent of claim 1, wherein the linker is a peptide, an aminohexonic acid, or a polyethylene glycol.

7. (canceled)

8. The reagent of claim 1, wherein the linker comprises the amino acid sequence (GGGSK) set forth in SEQ ID NO: 3, the amino acid sequence (GGGAGGG) set forth in SEQ ID NO: 28, or the amino acid sequence (GGGAGGGK) set forth in SEQ ID NO: 29.

9. The reagent of claim 6, wherein the polyethylene glycol is triethylene glycol (PEG3 or E3).

10. The reagent of claim 1 further comprising at least one detectable label attached to the peptide or attached to the peptide via the linker.

11. (canceled)

12. The reagent of claim 10, wherein the label is fluorescein isothiocyanate (FITC), Cyanine 5 (Cy5), Cyanine 5.5 (Cy5.5), or near-infrared (NIR) fluorescent dye 800 (IRDye800).

13-15. (canceled)

16. The reagent of claim 1, wherein at least one therapeutic moiety is attached to the heterodimeric peptide or a peptide monomer of the heterodimeric peptide.

17. The reagent of claim 16, wherein the therapeutic moiety is a chemotherapeutic agent.

18. The reagent of claim 16, wherein the therapeutic moiety is a micelle or provided in a micelle.

19. The reagent of claim 18, wherein the micelle is an octadecyl lithocholate micelle.

20. The reagent of claim 18, wherein the micelle is pegylated.

21. (canceled)

22. The reagent of claim 18, wherein the micelle comprises trastuzumab or ramucirumab.

23. A composition comprising the reagent of claim 1 and a pharmaceutically acceptable excipient.

24. A method for detecting esophageal adenocarcinoma (EAC), high grade dysplasia (HGD) of the esophagus, or Barrett's neoplasia in a patient comprising the steps of administering the reagent of claim 1 to the patient and detecting binding of the reagent to esophageal cells of the patient.

25. A method of determining the effectiveness of a treatment for esophageal adenocarcinoma (EAC), high grade dysplasia (HGD) of the esophagus, or Barrett's neoplasia in a patient comprising the step of administering the reagent of claim 1 to the patient, visualizing a first amount of cells labeled with the reagent, and comparing the first amount to a previously-visualized second amount of cells labeled with the reagent,
wherein a decrease in the first amount cells labeled relative to the previously-visualized second amount of cells labeled is indicative of effective treatment.

26. (canceled)

27. A method for delivering a therapeutic moiety to esophageal adenocarcinoma (EAC) cells, high grade dysplastic (HGD) cells of the esophagus, or Barrett's neoplastic cells of a patient comprising the step of administering the reagent of claim 1 to the patient.

28. A kit comprising the composition of claim 23 and instructions for use of the composition in a patient or cells of a patient.

29. (canceled)


SEQUENCE LISTING (29 sequences)

SEQ ID NO: 1 - protein, 7 aa, synthetic construct
  REGION 1..7: note = Synthetic Polypeptide
QRHKPRE

SEQ ID NO: 2 - protein, 7 aa, synthetic construct
  REGION 1..7: note = Synthetic Polypeptide
KSPNPRF

SEQ ID NO: 3 - protein, 5 aa, synthetic construct
  REGION 1..5: note = Synthetic Polypeptide
GGGSK

SEQ ID NO: 4 - protein, 7 aa, synthetic construct
  REGION 1..7: note = Synthetic Polypeptide
PPSNFKR

SEQ ID NO: 5 - protein, 12 aa, synthetic construct
  REGION 1..12: note = Synthetic Polypeptide
NRHKPREKTFTD

SEQ ID NO: 6 - protein, 6 aa, synthetic construct
  REGION 1..6: note = Synthetic Polypeptide
PEHKRR

SEQ ID NO: 7 - protein, 16 aa, synthetic construct
  REGION 1..16: note = Synthetic Polypeptide
GGGAGGGGGGAGGGKK

SEQ ID NO: 8 - DNA, 20 bp, synthetic construct
  misc_feature 1..20: note = Synthetic polynucleotide
  source 1..20: mol_type = other DNA
ccctcatagttagcgtaacg

SEQ ID NO: 9 - protein, 7 aa, synthetic construct
  REGION 1..7: note = Synthetic Polypeptide
HAHRSWS

SEQ ID NO: 10 - protein, 7 aa, synthetic construct
  REGION 1..7: note = Synthetic Polypeptide
YLTMPTP

SEQ ID NO: 11 - protein, 7 aa, synthetic construct
  REGION 1..7: note = Synthetic Polypeptide
TYPISFM

SEQ ID NO: 12 - protein, 7 aa, synthetic construct
  REGION 1..7: note = Synthetic Polypeptide
KLPGWSG

SEQ ID NO: 13 - protein, 7 aa, synthetic construct
  REGION 1..7: note = Synthetic Polypeptide
IQSPHFF

SEQ ID NO: 14 - protein, 7 aa, synthetic construct
  REGION 1..7: note = Synthetic Polypeptide
YSIPKSS

SEQ ID NO: 15 - protein, 12 aa, synthetic construct
  REGION 1..12: note = Synthetic Polypeptide
SHRNRPRNTQPS

SEQ ID NO: 16 - protein, 12 aa, synthetic construct
  REGION 1..12: note = Synthetic Polypeptide
TAVPLKRSSVTI

SEQ ID NO: 17 - protein, 12 aa, synthetic construct
  REGION 1..12: note = Synthetic Polypeptide
GHTANRQPWPND

SEQ ID NO: 18 - protein, 12 aa, synthetic construct
  REGION 1..12: note = Synthetic Polypeptide
LSLTRTRHRNTR

SEQ ID NO: 19 - protein, 12 aa, synthetic construct
  REGION 1..12: note = Synthetic Polypeptide
RHRDTQNHRPTN

SEQ ID NO: 20 - protein, 12 aa, synthetic construct
  REGION 1..12: note = Synthetic Polypeptide
ARHRPKLPYTHT

SEQ ID NO: 21 - protein, 12 aa, synthetic construct
  REGION 1..12: note = Synthetic Polypeptide
KRPRTRNKDERR

SEQ ID NO: 22 - protein, 12 aa, synthetic construct
  REGION 1..12: note = Synthetic Polypeptide
SPMPQLSTLLTR

SEQ ID NO: 23 - protein, 12 aa, synthetic construct
  REGION 1..12: note = Synthetic Polypeptide
NHVHRMHATPAY

SEQ ID NO: 24 - protein, 7 aa, synthetic construct
  REGION 1..7: note = Synthetic Polypeptide
RHPFPRF

SEQ ID NO: 25 - protein, 7 aa, synthetic construct
  REGION 1..7: note = Synthetic Polypeptide
RHPWPNR

SEQ ID NO: 26 - protein, 7 aa, synthetic construct
  REGION 1..7: note = Synthetic Polypeptide
RHPYPQR

SEQ ID NO: 27 - protein, 7 aa, synthetic construct
  REGION 1..7: note = Synthetic Polypeptide
RKPFPRH

SEQ ID NO: 28 - protein, 7 aa, synthetic construct
  REGION 1..7: note = Synthetic Polypeptide
GGGAGGG

SEQ ID NO: 29 - protein, 8 aa, synthetic construct
  REGION 1..8: note = Synthetic Polypeptide
GGGAGGGK